(19) United States
(12) Patent Application Publication
(10) Pub. No.: US 2023/0100616 A1
(43) Pub. Date: Mar. 30, 2023

(54) BIOMARKERS FOR DETECTING OF OUTCOME/RISK OF THE PATIENTS WITH A RESPIRATORY ILLNESS

(71) Applicant: UNIVERSITY HEALTH NETWORK, Toronto (CA)

(72) Inventors: Andrew Sage, Toronto (CA); Jerome Valero, Whitby (CA); Lorenzo Del Sorbo, Toronto (CA); Marcelo Cypel, Toronto (CA); Bo Wang, Toronto (CA); Shafique Keshavjee, Toronto (CA)

(21) Appl. No.: 17/911,208

(22) PCT Filed: Mar. 15, 2021
(86) PCT No.: PCT/CA2021/050343

§ 371 Date: Sep. 13, 2022

Related U.S. Application Data
(60) Provisional application No. 62/989,276, filed on Mar. 13, 2020.

(51) Int. Cl. G01N 33/68; G16H 50/30

(52) U.S. Cl. CPC G01N 33/6863; G16H 50/30; G01N 2800/125; G01N 2800/52; G01N 2333/715

(57) ABSTRACT

Methods and kits for screening, diagnosing, detecting or predicting a patient outcome/risk in a patient with a respiratory illness, the method comprising: a. obtaining a sample obtained from the patient; b. quantitatively measuring in the sample a polypeptide level of one or more biomarkers selected from: IL-6, CXCL8, IL-10, IL-IRA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α, VEGF, sTNFR1 and sTREM1; and c. i) comparing the level of the one or more biomarkers in the sample with a control or cut-off level, wherein the differential level is indicative of patient outcome risk; or ii) using the polypeptide level of several of the biomarkers in combination, as inputs for an algebraic calculation or machine learning model of patient outcome risk.

### CROSS-REFERENCE TO RELATED APPLICATION

This PCT application claims the benefit of United States Provisional Patent Applications U.S. 62/989,276 filed Mar. 13, 2020 herein incorporated by reference.

### FIELD

The disclosure pertains to biomarkers, methods, immunoassays and kits for assessing blood samples of patients afflicted with a respiratory illness for predicting patient outcome.

### BACKGROUND

Depending on a variety of factors, infections that affect the respiratory tract, can be mild to life-threatening. Recent epidemics such as severe acute respiratory syndrome coronavirus (SARS-CoV), Middle East respiratory syndrome coronavirus (MERS-CoV) and most recently a novel severe acute respiratory syndrome coronavirus named SARS-nCoV-2, have together caused more than 100,000 cumulative cases in the past two decades. The mortality rate for SARS-CoV has been estimated to be about 10%, for MERS-CoV about 37% and for SARS-nCoV-2 about 3% although this may be an underestimation.

Huang et al 2020 reported that SARS-nCoV-2 infection caused severe respiratory illness similar to SARS-CoV and was associated with ICU admission and high mortality.

Compared with non-ICU patients, ICU patients had higher plasma levels of IL-2, IL-7, IL-10, GSCF, IP10, MCP1, MIP1A and TNFa (1).

Biomarkers and methods for stratification of infected patients and determination of the outcome or risk of patients developing respiratory distress are desirable.

### SUMMARY

The disclosure provides in an aspect, methods for determining patient outcome (PO) risk in a patient with a respiratory illness. Also provided in other aspects are methods and systems for determining if a patient, for example, presenting in an emergency department, should be hospitalized or can be safely discharged.

The method can for example be used to screen or stratify patients with respiratory distress according to risk of illness severity for prioritizing access to hospitalization, mechanical ventilation, ICU treatment etc.

An aspect of the disclosure is a method for determining patient outcome risk in a patient with a respiratory illness, the method comprising:
a) obtaining a sample obtained from the patient;
b) quantitatively measuring in the sample a polypeptide level of one or more, preferably two or more, biomarkers selected from: sTNFR1, sTREM1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α or VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10; and
   wherein the level of the one or more biomarkers in the sample determines patient outcome risk of the patient.

Another aspect of the disclosure is a method for determining patient outcome risk in a patient with a respiratory illness, the method comprising:
a) obtaining a sample obtained from the patient;
b) quantitatively measuring in the sample a polypeptide level of one or more, preferably two or more, biomarkers selected from: sTNFR1, sTREM1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF a or VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10; and
c) i) comparing the level of the one or more biomarkers in the sample with a control or cut-off level, wherein the differential level is indicative of patient outcome risk; or
   ii) using the polypeptide level of several of the biomarkers in combination, as inputs for an algebraic calculation or machine learning model to determine patient outcome risk.

In one embodiment, where respiratory illness is acute respiratory distress syndrome (ARDS) related to an infection.

In another embodiment, the infection is Influenza A, optionally influenza A is subtype H1N1.

In a further embodiment, the infection is Influenza B.

In yet another embodiment, the infection is a coronavirus infection, optionally wherein the coronavirus is SARS-CoV, MERS-CoV or the coronavirus is SARS-nCoV-2019.

In another embodiment, the infection is a bacterial pneumonia.

In a further embodiment, the respiratory illness is ARDS related to trauma.

In another embodiment, respiratory distress is ARDS related to exposure to an exogenous substance.

In a further embodiment, the sample is whole blood, optionally wherein the sample is processed to obtain plasma prior to the measuring step.

In yet another embodiment, the sample is plasma.

In another embodiment, the sample is serum.

In another embodiment, the level of sTNFR1, sTREM1, and IL-6; sTNFR1, sTREM1, IL-6 and IL-8; or sTNFR1, sTREM1, IL-6, IL-8 and IL-10 is measured.

In another embodiment, the level of at least 2 or 3 biomarkers is measured.

In a further embodiment the level of at least 4 biomarkers is measured.

In another embodiment, the level of at least 5 biomarkers is measured.

In yet another embodiment, the method further comprises determining a CRB-65 score and using said score as a further input in the algebraic calculation or machine learning algorithm in determine the patient outcome risk.

In another embodiment, the level of at least 2 biomarkers up to all of the biomarkers of the disclosure is measured.

In a further embodiment, wherein the level of IL-6, IL-8, IL-10, sTREM1, sTNFR1 is measured

In another embodiment, the patient outcome risk is:

requirement of hospitalization or safe discharge,

requirement of mechanical ventilation,

requirement of treatment in an intensive care unit (ICU), and/or increased risk of death.

In another embodiment, the patient outcome risk is requirement for hospitalization or safe discharge and the method further comprises hospitalizing the patient or discharging the patient according to the patient outcome risk.

In a further embodiment, the patient outcome risk is requirement for ventilation, and the method further comprises mechanically ventilating the patient.

In another embodiment, the patient outcome risk is requirement for treatment in the ICU and the method further comprises treating the patient in the ICU.

In yet another embodiment, the sample is obtained from a patient that is hospitalized.

In another embodiment, the patient in hospital is obtained after the patient has a change in one or more symptoms of the respiratory illness.

In another embodiment the change is amelioration of one or more symptoms of the respiratory illness and the patient is assessed for safe discharge.

In a further embodiment, the change is worsening of one or more symptoms of the respiratory illness and the patient is assessed for requirement for mechanical ventilation or treatment in the ICU.

In another embodiment, the method further comprises discharging the patient when the patient is determined to be safe to discharge or the method further comprises mechanically ventilating the patient and/or treating the patient in the ICU when the patient is determined to require mechanical ventilation and/or ICU treatment.

Another aspect of the disclosure is a method for triaging a patient with a respiratory illness, the method comprising:
a) obtaining a sample obtained from the patient;
b) quantitatively measuring in the sample a polypeptide level of two or more biomarkers, the biomarkers comprising sTNFR1 and sTREM1, and optionally one or more of IL-6, IL-8 and IL-10, preferably wherein the two or more markers are sTNFR1, sTREM1, IL-6, IL-8 and IL-10; and
c) i) comparing the level of the two or more biomarkers in the sample with a control or cut-off level, wherein the differential level is indicative of patient outcome risk; or
   ii) using the polypeptide level of several of the biomarkers in combination, as inputs for an algebraic calculation or machine learning model to determine whether the patient should be hospitalized or can be safely discharged.

In an embodiment, respiratory illness is acute respiratory distress syndrome (ARDS) related to an infection.

In another embodiment, the infection is Influenza A, optionally influenza A is subtype H1N1.

In another embodiment, the infection is Influenza A, optionally influenza A is subtype H1N1.

In another embodiment, the infection is Influenza B.

In a further embodiment, the infection is a coronavirus infection, optionally wherein the coronavirus is SARS-CoV, MERS-CoV or the coronavirus is SARS-nCoV-2019.

In another embodiment, the infection is a bacterial pneumonia.

In another embodiment, the respiratory illness is ARDS related to trauma.

In yet another embodiment, respiratory distress is ARDS related to exposure to an exogenous substance.

In another embodiment, the sample is whole blood and optionally the sample is processed to obtain plasma prior the measuring step.

In another embodiment, the sample is plasma.

In another embodiment, the sample is serum.

In yet another embodiment, the level of sTNFR1, sTREM1, and IL-6; sTNFR1, sTREM1, IL-6 and IL-8; or sTNFR1, sTREM1, IL-6, IL-8 and IL-10 is measured.

In a further embodiment, the level of at least 3 biomarkers is measured.

In another embodiment, the level of at least 4 biomarkers is measured.

In another embodiment, the level of at least 5 biomarkers is measured.

In a further embodiment, the method further comprises determining a CRB-65 score and using said score as a further input in the algebraic calculation or machine learning algorithm in determining whether the patient should be hospitalized or can be safely discharged.

In another embodiment, the level of at least 2 biomarkers up to all of the biomarkers of the disclosure is measured.

In another embodiment, the level of IL-6, IL-8, IL-10, sTREM1, sTNFR1 is measured.

In a further embodiment, the method further comprises hospitalizing the patient or discharging the patient.

In yet another embodiment, the sample is obtained upon clinical presentation, optionally at an emergency room or urgent care centre.

In another embodiment, the sample is obtained from a patient in hospital.

In another embodiment, the polypeptide level of the one or more, preferably two or more, biomarkers is measured using a multiplex assay, optionally a 5-plex assay.

In another embodiment, the quantitively measuring comprises the steps of incubating the sample with a detection agent for each of the one or more, preferably two or more, biomarkers; obtaining signal intensities for each of the one or more, preferably two or more, biomarkers, processing the signal intensities to calculate concentrations of the one or more, preferably two or more, biomarkers in the sample, wherein the concentrations are compared or used as inputs in the method.

In yet another embodiment, the machine learning model comprises a decision tree.

In another embodiment, the polypeptide level is measured using an assay or kit with a limit of detection for each of the one or more, preferably two or more, biomarkers, wherein the lower limit of detection (LLOD) at least 1 pg/mL for IL-6, IL-8, and/or IL-10 and at least 15 pg/mL for sTNFR1 and/or sTREM1.

In yet another embodiment, the LLOD for IL-6 is at least 21 pg/mL, for IL-8 is at least 27 pg/mL, for IL-10 is at least 7 pg/mL, for sTNFR1 is at least 17 pg/mL, and/or for sTREM1 is at least 44 pg/mL.

In another embodiment, the relative feature importance of the biomarkers of the machine learning model can be given by their Shapley Additive Explanations (SHAP) values.

Another embodiment is for screening or stratifying patients as less or more likely to require hospitalization, mechanical ventilation and/or ICU treatment or the method described herein for screening patients as less or more likely to require hospitalization or to be less or more likely to be safely discharged.

Another aspect of the disclosure is a kit or immunoassay comprising at least a detection antibody specific for sTNFR1 and a detection antibody specific for sTREM1 and optionally one or more other detection antibodies each specific for a biomarker selected from IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α and VEGF.

In one embodiment, the one or more detection antibodies comprise antibodies specific for IL-6, IL-8 and IL-10.

In another embodiment, the detection antibodies are coupled to beads and/or labelled.

Another embodiment further comprises one or more of a 96-well plate, optionally wherein the detection antibodies are fixed, standards, assay buffer, wash buffer, sample diluent, standard diluent, detection antibody diluent, streptavidin-PE, a filter plate or sealing tape.

Another embodiment is a kit or immunoassay for performing the method described herein.

A further aspect of the disclosure provides a computer-implemented method for determining patient outcome risk in a patient with a respiratory illness, the method comprising:
obtaining a polypeptide level of one or more, preferably two or more, biomarkers selected from: sTNFR1 and sTREM-1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α and/or VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10;
calculating patient outcome risk with a machine learning model using the received polypeptide levels as inputs.

In one embodiment, the respiratory illness is acute respiratory distress syndrome (ARDS) related to an infection.

In another embodiment, the infection is Influenza A, optionally influenza A is subtype H1N1.

In another embodiment, the infection is Influenza B.

In another embodiment, the infection is a coronavirus infection, optionally wherein the coronavirus is SARS-CoV, MERS-CoV or the coronavirus is SARS-nCoV-2019.

In a further embodiment, the infection is a bacterial pneumonia.

In another embodiment, the respiratory illness is ARDS related to trauma.

In another embodiment, the respiratory distress is ARDS related to exposure to an exogenous substance.

In yet another embodiment, the patient outcome risk is: requirement of hospitalization, requirement of mechanical ventilation, requirement of treatment in the intensive care unit (ICU), and/or increased risk of death.

In another embodiment, the step of obtaining a polypeptide level method further includes the step of: quantitatively measuring a polypeptide level of one or more, preferably two or more, biomarkers of a sample obtained from a patient.

In a further embodiment, the biomarkers selected include IL-6, IL-8, IL-10, sTNFR1 and sTREM1, and optionally comprises using a CRB-65 score as an input.

In another embodiment, the machine learning model comprises a decision tree.

In a further embodiment, the relative feature importance of the biomarkers of the machine learning model can be given by their Shapley Additive Explanations (SHAP) values.

Another aspect of the disclosure provides a system for determining patient outcome risk in a patient with a respiratory illness, the system comprising:
a processor; and
at least one non-transitory memory containing instructions which when executed by the processor cause the system to:
obtain a polypeptide level of one or more, preferably two or more, biomarkers selected from: sTNFR1 and sTREM-1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α, and VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10, and optionally a CRB-65 score; and
calculate patient outcome risk with a machine learning model using the received polypeptide levels and optionally CRB-65 score as inputs.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

Embodiments of the disclosure are described with reference to the drawings:

FIG. 1 is a schematic showing patient enrollment and initial ED triage decision to hospitalize patients in the ETC-19 Study;

FIG. 2 is a series of Box and Whisker plots for IL-6, IL-8, IL-10, sTNFR1, and sTREM1 separated by 28-day mortality, requirement of care in the ICU, severity of COVID-19, or development of COVID-19 pneumonia. Mann-Whitney U (mortality, ICU care, COVID-19 pneumonia) and Kruskal-Wallis (COVID-19 severity) test p-values are indicated above each panel (*p<0.05, **p<0.01);

FIG. 3 is a schematic providing the multivariate model performance (AUROC) of the panel biomarkers ED triage models (panel biomarkers, panel biomarkers+CRB-65, All Variable) in the development (ETC-19) and validation (Italy) cohorts;

FIG. 4 is a series of Box and Whisker plots for IL-6, IL-8, IL-10, sTNFR1, and sTREM1 separated by gender, age, ethnicity, or BMI. Mann-Whitney U (gender, BMI) and Kruskal-Wallis (age, ethnicity) test p-values are indicated in each panel (*p<0.05, **p<0.01, ****p<0.001);

FIG. 5 is a series of Box and Whisker plots for IL-6, IL-8, IL-10, sTNFR1, and sTREM1 separated by heart rate, temperature, oxygen saturation, or CRB-65 score. Mann-Whitney U (heart rate, temperature, CRB-65 score) and Kruskal-Wallis (oxygen saturation) test p-values are indicated in each panel (*p<0.05, **p<0.01, ****p<0.001);

FIG. 6 is a series of Box and Whisker plots for IL-6, IL-8, IL-10, sTNFR1, and sTREM1 separated by bacteremia, community acquired pneumonia (CAP), acute respiratory distress syndrome (ARDS), and COVID-19. Mann-Whitney U test p-values are indicated above each graph (*p<0.05, **p<0.01, ****p<0.001);

FIG. 7 is a series of Box and Whisker plots for patients that were discharged or hospitalized following ED presentation for: IL-6, IL-8, IL-10, sTNFR1, and sTREM1. Mann-Whitney U test p-values are indicated within each graph (*p<0.05, **p<0.01, ****p<0.001);

FIG. 8 is a series of Box and Whisker plots for IL-6, IL-8, IL-10, sTNFR1, and sTREM1 separated by patients that spent +/−72-hours in hospital following ED admission. Mann-Whitney U test p-values are indicated above each graph (*p<0.05, **p<0.01);

FIG. 9 is a series of Box and Whisker plots for patients that were discharged or hospitalized following ED presentation for: IL-6, IL-8, IL-10, sTNFR1, and sTREM1. Mann-Whitney U test p-values are indicated above each graph (*p<0.05, **p<0.01, ****p<0.001);

FIG. 10 is a schematic diagram of an example of a computing environment; and

FIG. 11 is a flow chart of computer implemented method 1100.

### DETAILED DESCRIPTION OF THE DISCLOSURE

### I. Definitions

The term “patient outcome” also referred to as “outcome” as used herein means one or more of but not limited to requirement for hospitalization, safe discharge or total hospital length of stay, or for example need intensive care unit (ICU) treatment, total ICU length of stay, requirement for mechanical ventilation, optionally invasive or non-invasive ventilation, days on mechanical ventilation, requirement for intubation and mechanical ventilation and/or patient death. A patient who is predicted to have a high risk of needing hospitalization (i.e. a patient who could not be safely discharged) would be slated to be hospitalized. A patient who has a patient outcome risk requiring ICU or mechanical ventilation for example is a patient who would require hospitalization and/or such treatment. Accordingly, the methods can be used to assess if a patient needs hospitalization and/or for particular requirements, e.g. requirement for ICU treatment and/or mechanical ventilation.

The term “biomarkers of the disclosure” as used herein means one or more, preferably two or more, of sTREM1 (soluble (TREM1)), sTNFR1 (soluble (TNFRSF1A)), IL-6 (IL6), IL-8 (CXCL8), IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α, and VEGF.

The term “panel biomarkers” or “Panel” as used herein means biomarkers sTREM1 (soluble (TREM1)), sTNFR1 (soluble (TNFRSF1A)), IL-6 (IL6), IL-8 (CXCL8), and IL-10.

The term “polypeptide” as used herein refers to a polymer consisting a number of amino acid residues bonded together in a chain. The polypeptide can form a part or the whole of a protein. The polypeptide may be arranged in a long, continuous and unbranched peptide chain. The polypeptide may also be arranged in a biologically functional way. The polypeptide may be folded into a specific three dimensional structure that confers it a defined activity. The term “polypeptide” as used herein is used interchangeably with the term “protein”.

The term “soluble TREM1” or sTREM1 as used herein means non-cell bound forms of Triggering receptor expressed on myeloid cells and includes all naturally occurring cleaved or released forms, for example from all species and particularly human including for example human sTREM1 which has at least the extracellular portion of sTREM1, for example amino acid 21 to 205 of accession number Q9NP99, herein incorporated by reference.

The term “IL-6” or “IL6” as used herein means interleukin-6 which is a secreted cytokine, and includes all naturally occurring forms, for example from all species and particularly human including for example human IL-6 which has amino acid sequence accession P05231, herein incorporated by reference.

The term “IL-8” also referred to as CXCL8, as used herein means interleukin-8 which is a secreted cytokine, and includes all naturally occurring forms, for example from all species and particularly human including for example human IL-8 which has amino acid sequence accession P10145, herein incorporated by reference.

The term “sTNFR1” or “soluble (TNFRSF1A)” used herein means non-cell bound forms of tumor necrosis factor (TNF) receptor superfamily member 1A, and includes all naturally occurring cleaved or released forms, for example from all species and particularly human including for example human sTNFR1 which has at least the extracellular portion of TNFR1, for example amino acid 22 to 211 of accession number P19438, herein incorporated by reference.

The term “IL-10” as used herein means interleukin 10 and includes all naturally occurring forms, for example from all species and particularly human including for example accession number P22301 herein incorporated by reference.

The term “IL-1RA” as used herein means interleukin 1 receptor antagonist and includes all naturally occurring forms, for example from all species and particularly human including for example human IL-1RA which has amino acid sequence accession P14778 herein incorporated by reference.

The term “IL-2” as used herein means interleukin 2 and includes all naturally occurring forms, for example from all species and particularly human including for example human IL-2 which has amino acid sequence accession P60568 herein incorporated by reference.

The term “IL-4” as used herein means interleukin 4 and includes all naturally occurring forms, for example from all species and particularly human including for example human IL-4 which has amino acid sequence accession P05112 herein incorporated by reference.

The term “IL-7” as used herein means interleukin 7 and includes all naturally occurring forms, for example from all species and particularly human including for example human IL-7 which has amino acid sequence accession P13232 herein incorporated by reference.

The term “IL-9” as used herein means interleukin 9 and includes all naturally occurring forms, for example from all species and particularly human including for example human IL-9 which has amino acid sequence accession P15248 herein incorporated by reference.

The term “IL-13” as used herein means interleukin 13 and includes all naturally occurring forms, for example from all species and particularly human including for example human IL-13 which has amino acid sequence accession P35225 herein incorporated by reference.

The term “IL-17” as used herein means interleukin 17 and includes all naturally occurring forms, for example from all species and particularly human including for example human IL-17 which has amino acid sequence accession Q16552, Q9UHF5, Q9P0M4, Q8TAD2, Q8NFR9, Q96PD4 herein incorporated by reference.

The term “IFN-γ” as used herein means interferon gamma and includes all naturally occurring forms, for example from all species and particularly human including for example human IFN-γ which has amino acid sequence accession herein incorporated by reference.

The term “IP-10” also known as C-X-C motif chemokine 10 (CXCL10) as used herein means interferon gamma inducible protein 10 kDa and includes all naturally occurring forms, for example from all species and particularly human including for example human IP-10 which has amino acid sequence accession P02778 herein incorporated by reference.

The term “MCP-1” as used herein means Monocyte chemoattractant protein-1 and includes all naturally occurring forms, for example from all species and particularly human including for example human MCP-1 which has amino acid sequence accession P13500 herein incorporated by reference.

The term “G-CSF” as used herein means granulocyte colony stimulating factor and includes all naturally occurring forms, for example from all species and particularly human including for example human G-CSF which has amino acid sequence accession P09919 herein incorporated by reference.

The term “GM-CSF” as used herein means granulocyte macrophage colony stimulating factor also known as colony-stimulating factor 2 (CSF2) and includes all naturally occurring forms, for example from all species and particularly human including for example human GM-CSF which has amino acid sequence accession herein incorporated by reference.

The term “FGF-basic” as used herein means basic fibroblast growth factor, also known as FGF2 and bFGF, and includes all naturally occurring forms, for example from all species and particularly human including for example human FGF-basic which has amino acid sequence accession P09038 herein incorporated by reference.

The term “SCGF-β” as used herein means stem cell growth factor beta, and includes all naturally occurring forms, for example from all species and particularly human including for example human SCGF-β which has amino acid sequence accession Q9Y240 herein incorporated by reference.

The term “Gro-α” as used herein means growth regulated oncogene alpha, also known as CXCL1, and includes all naturally occurring forms, for example from all species and particularly human including for example human Gro-α which has amino acid sequence accession P09341 herein incorporated by reference.

The term “MIP-1α” as used herein means macrophage inflammatory protein alpha, also known as CCL3, and includes all naturally occurring forms, for example from all species and particularly human including for example human MIP-1α which has amino acid sequence accession P101747 herein incorporated by reference.

The term “MIP-1 β” as used herein means macrophage inflammatory protein alpha, also known as CCL4, and includes all naturally occurring forms, for example from all species and particularly human including for example human MIP-1 β which has amino acid sequence accession P13236 herein incorporated by reference.

The term “CK-18” as used herein means cytokeratin-18 and includes all naturally occurring forms, for example from all species and particularly human including for example human CK-18 which has amino acid sequence accession P05783] herein incorporated by reference.

The term “PDGF-bb” as used herein means platelet derived growth factor composes of two B subunits and includes all naturally occurring forms, for example from all species and particularly human including for example human PDGF-bb which has amino acid sequence accession P01127 herein incorporated by reference.

The term “caspase 3” as used herein includes all naturally occurring forms, for example from all species and particularly human including for example human caspase 3 which has amino acid sequence accession P42574 herein incorporated by reference.

The term “HMGB-1” as used herein means High mobility group box 1 protein and includes all naturally occurring forms, for example from all species and particularly human including for example human HMGB-1 which has amino acid sequence accession P09429 herein incorporated by reference.

The term “TNF-α” as used herein means tumor necrosis factor alpha and includes all naturally occurring forms, for example from all species and particularly human including for example human TNF-α which has amino acid sequence accession P01375 herein incorporated by reference.

The term “VEGF” as used herein means vascular endothelial growth factor and includes all naturally occurring forms, for example from all species and particularly human including for example human VEGF which has amino acid sequence accession P15692 herein incorporated by reference.

The terms “control” and “cut-off level” as used herein respectively refer to a control patient such as a healthy patient or patient with known outcome and a predetermined threshold value based on a plurality of known outcome patients, and for biomarkers associated with increased polypeptide level in poor PO, above which threshold a patient is identified as having an increased risk of developing poor PO and below which (and/or comparable to) a patient is identified as having a decreased risk of developing poor PO. The threshold value can for example for each of the one or more polypeptide biomarkers of the disclosure, be determined from the levels related thereto of the biomarkers in a plurality of known outcome patients. For example, an optimal or an acceptable threshold can be selected based on the desired tolerable level of risk. The cut-off level may, for example, depend on the PO being assessed. The cut-off level may also for example include patient characteristics, for example gender, underlying disease such as diabetes, hypertension or cardiovascular disease, age, body mass index (BMI), and/or smoking history. Accordingly, the biomarker ‘cut-off’ can in some embodiments be adjusted for patients with underlying disease.

The term “good outcome patient” as used herein means patient that is predicted to not need or less likely to need hospitalization (e.g. can be safely discharged), mechanical ventilation or ICU treatment to recover from their respiratory illness and/or has a decreased risk of death.

The term “safely dischargeable” or “safely discharged” as used herein refers to a patient that can be or is discharged and is unlikely to need further care or is an avoidable admission.

The term “avoidable admission” as used herein refers to a patient admitted to hospital and discharged from hospital without requiring significant medical intervention related to the admission condition, for example, without requiring invasive intervention (e.g. surgery or percutaneous procedures), without requiring regional or general anesthesia, without requiring I.V. treatment for neurological, respiratory or hemodynamic disorders and/or without requiring supplemental oxygen. An avoidable admission includes for example patients discharged within 72 hours without requiring significant medical intervention.

The term “poor outcome patient” as used herein means a patient that is predicted to need or more likely to need one or more of hospitalization, mechanical ventilation or ICU treatment to recover from their respiratory illness and/or has an increased risk of death.

The term “antibody” as used herein is intended to include monoclonal antibodies including chimeric and humanized monoclonal antibodies, polyclonal antibodies, humanized antibodies, human antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques. The skilled person can readily recognize that a suitable antibody is any antibody useful for detecting biomarkers described herein in any detection method described herein. For example, useful antibodies include antibodies that specifically bind to a biomarker of the disclosure described herein.

The term “detection agent” refers to an agent (optionally a detection antibody) that selectively binds and is capable of binding its cognate biomarker compared to another molecule and which can be used to detect a level and/or the presence of the biomarker. A biomarker specific detection agent can include probes and the like as well as binding polypeptides such as antibodies which can for example be used with immunohistochemistry (IHC), Luminex® based assays, ELISA, immunofluorescence, radioimmunoassay, dot blotting, FACS, protein microarray, Western blots, immunoprecipitation followed by SDS-PAGE immunocytochemistry Simple Plex assay or Mass Spectrometry to detect the polypeptide level of a biomarker described herein. Similarly, “an antibody or fragment thereof” (e.g. binding fragment), that specifically binds a biomarker refers to an antibody or fragment that selectively binds its cognate biomarker compared to another molecule. “Selective” is used contextually, to characterize the binding properties of an antibody. An antibody that binds specifically or selectively to a given biomarker or epitope thereof will bind to that biomarker and/or epitope either with greater avidity or with more specificity, relative to other, different molecules. For example, the antibody can bind 3-5, 5-7, 7-10, 10-15, 5-15, or 5-30 fold more efficiently to its cognate biomarker compared to another molecule. The “detection agent” or detection antibody can, for example, be coupled to or couplable to (e.g. via a secondary antibody) or labeled with a detectable label. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The term “level” as used herein refers to an amount (e.g. relative amount or absolute concentration) of biomarker (i.e. polypeptide related level) that is detectable, measurable or quantifiable in a test biological sample and/or a reference biological sample, for example, a test sample and/or a reference sample. For example, the level can be a rate such as pg/mL/hour, a concentration such as pg/L, ng/mL or pg/mL, a relative amount or ratio. The level of indicative of patient outcome can, for example, be 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 8, 9, 10, 11, 12, 13, 15, 20, 25, and/or 30 times more or less than a control biomarker level or above or below a cut-off level. The control biomarker polypeptide level cut-off level can, for example, be derived from the average or median level in a plurality of known outcome patients and/or healthy controls.

The term “subject” as used herein includes all members of the animal kingdom including mammals, and suitably refers to humans.

The term “symptoms of respiratory illness” includes but is not limited to difficulty breathing, increased breathing rate, decreased blood oxygen saturation, cough, sore throat, loss of taste, loss of smell, dyspnea, chest pain, fever, myalgia, and/or fatigue

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Methods

Disclosed herein are polypeptide biomarkers that can be used to assess whether a patient with a respiratory illness is likely to progress to respiratory distress. Such patients can for example be screened to determine their likely outcome risk and be stratified according to risk for treatment.

An aspect of the disclosure is a method for determining patient outcome risk in a patient with a respiratory illness, the method comprising:
a. obtaining a sample obtained from the patient;
b. quantitatively measuring in the sample a polypeptide level of one or more, preferably two or more, biomarkers selected from: sTNFR1, sTREM1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α or VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10; and
   wherein the level of the one or more biomarkers in the sample determines patient outcome risk of the patient.

In some embodiments, the levels of the one or more, preferably two or more biomarkers are used as inputs to determine

Another aspect of the present disclosure is a method for the screening, diagnosing, or detecting patient outcome risk in a patient with a respiratory illness, the method comprising:
a. obtaining a biological sample obtained from the patient;
b. quantitatively measuring in the sample a polypeptide level of one or more or two or more biomarkers, the one or more or two or more biomarkers selected from: IL-6, CXCL8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α, VEGF, sTNFR1 and sTREM1; and
c. i) comparing the level of the one or more, preferably two or more, biomarkers in the sample with a control or cut-off level, wherein the differential level is indicative of patient outcome risk; or
   ii) using the polypeptide level of several of the biomarkers in combination, as inputs for an algebraic calculation or machine learning model of patient outcome risk, optionally calculating a patient outcome score (PO score).

In an embodiment, the patient outcome risk is selected from: requirement of hospitalization, requirement of mechanical ventilation, requirement of treatment in the intensive care unit (ICU), and/or increased risk of death.

In an embodiment, the patient outcome risk is safe discharge. For example, the methods and systems described herein can be used to determine the risk associated with discharge or need for hospitalization.

In an embodiment, the patient outcome risk can also be further delineated as length of hospitalization, mechanical ventilation, for example, number of days in hospital or on a ventilator, or ICU length of stay.

The PO score can for example include other variables for example patient parameters, such as underlying disease.

In an embodiment, the method further comprises identifying a patient that has a decreased risk of having poor PO or is likely to have a good outcome. For example, the patient may have a decreased risk of poor PO or likely to have a good outcome, if the level, combination of levels, PO-score is/are below the selected cut-off value.

The algebraic calculation or machine learning model is calculated using a programmed computer or specifically programmed device. For example, a software module can be integrated into a device that measures polypeptide levels such as a plate reader or point of care device, that is programmed to provide a determination (e.g. provide a readout or otherwise display) for example of PO e.g. patient safe to discharge or patient should be hospitalized. Any device with a CPU could be utilized.

In another embodiment, the method further comprises identifying a patient that has an increased risk of having a poor patient outcome if the if the level, combination of levels, PO-score is/are e is unacceptable or above the cut-off value. For example, the patient may be identified as needing hospitalization, mechanical ventilation or ICU stay, or having an increased risk of death, if the level, combination of levels, or score is/are above the cut-off value.

The sample can be collected from the patient upon presentation to a health assessment facility for example, during ER (or similar) screening visit. The sample obtained from the patient can then be sent for processing and/or measuring.

As demonstrated herein, the methods, kits and systems can be used for making an early determination such as at the time of presentation at an emergency room or at an urgent care centre. Such determinations can be made quickly and early thereby reducing backlog and hospital resources.

Also provided is a method of monitoring a patient with a respiratory illness wherein a subsequent sample is obtained and compared to a prior a sample, where an increase in the level of one or more of the biomarkers of the disclosure is indicative that the patient is likely to have a worsening patient outcome and a decrease in the level of one or more of the biomarkers of the disclosure is indicative that the patient is likely to have an improving patient outcome.

In one embodiment, the concentration of the one or more, preferably two or more biomarkers of the disclosure is measured.

As indicated herein an algebraic formula or machine learning model can be used.

Logistic regression analysis is useful for univariate or multivariate analysis where the outcome has only a limited number of possible values. The skilled person in the art can readily recognize that logistic regression analysis is useful when the response variable is categorical in nature, such as to hospitalize or not. In an embodiment, patient outcome is predicted by logistic regression analysis.

The respiratory illness can be any respiratory illness. For example, a subject that show symptoms of fever and/or onset of cough or difficulty breathing (e.g. symptoms of respiratory infection) may be a candidate for the methods described herein.

In one embodiment, the respiratory illness is acute respiratory distress syndrome (ARDS) related to an infection. For example, the infection can be viral, for example Influenza A, optionally influenza A is subtype H1N1 or Influenza B. The infection can be a coronavirus infection, optionally wherein the coronavirus is SARS-CoV, MERS-CoV or the coronavirus is SARS-nCoV-2019. The infection can also be bacterial, for example causing pneumonia.

The patient may or may not be diagnosed with a particular viral or bacterial infection but may for example show radiologic or other findings or symptoms such as being in respiratory distress, e.g. chest infiltrates etc.

In one embodiment, the respiratory illness is ARDS related to trauma such as a motor vehicle accident or related to exposure of an exogenous substance such as vaping.

In one embodiment, the biomarker level is used to predict PO wherein the prediction can be substratified based on patient characteristics, for example, gender, underlying disease, age, and/or smoking history. Alternatively, the PO score can incorporate one or more patient characteristics.

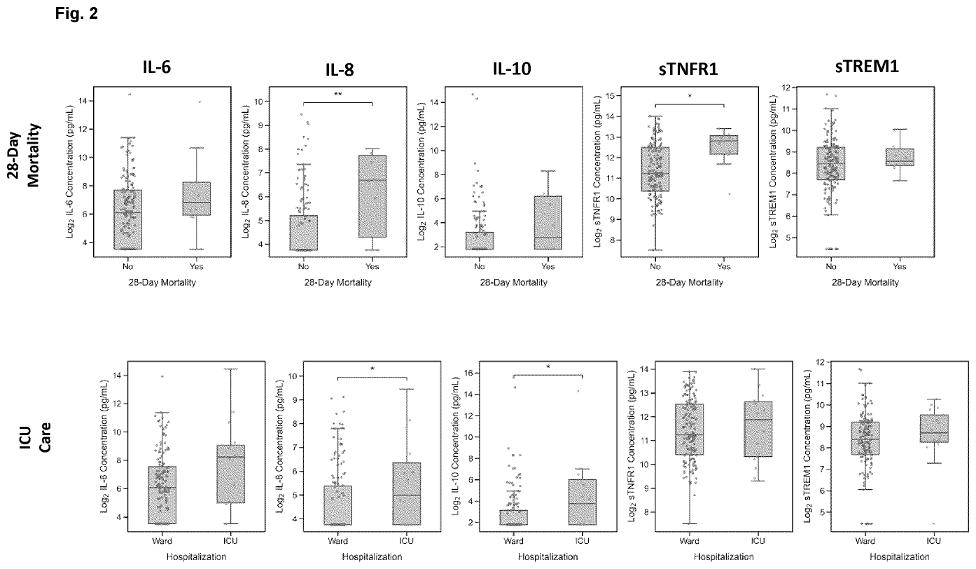

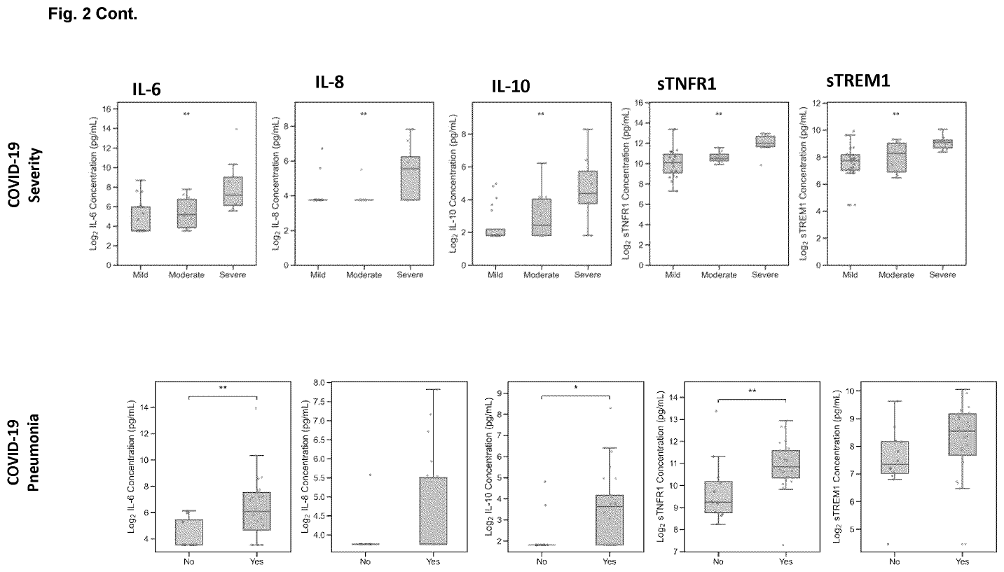

For example, the plasma levels of IL-8 and sTNFR1 were significantly elevated in patients that died during the 28-day follow-up period as shown in FIG. 2. Accordingly, patients with elevated plasma levels of IL-8 and sTNFR1 can be prioritized for and or treated in the ICU and/or with mechanical ventilation.

In some, embodiments, the method is used for predicting the type of mechanical ventilation required. In one embodiment, the type of ventilation is invasive. In another embodiment, the type of mechanical ventilation is non-invasive. The methods can also be used to assess patient outcome risk of intubation and mechanical ventilation. As shown for example in Table 7-8 the one or more biomarkers can be used assessing patient outcome regarding the type of mechanical ventilation (invasive vs. non-invasive). As further shown in Table 10, the one or more biomarker levels can be used to assess patient outcome risk such as the risk of intubation and mechanical ventilation, particularly for example in COVID-19 patients.

In one embodiment, the sample is whole blood, for example obtained by venous puncture and subsequently processed. The blood may be processed (e.g. by centrifuge) or used directly. In another embodiment, the sample is serum. In yet another embodiment, the sample is plasma.

In one embodiment, the level of at least 2 biomarkers is measured. In one embodiment, the level of at least 3 biomarkers is measured. In one embodiment, the level at least 4 biomarkers is measured. In one embodiment, the level of at least 5 biomarkers is measured. In one embodiment, the level of at least 6 biomarkers is measured. In one embodiment, the level of at least 7 biomarkers is measured. In one embodiment, the level of at least 8 biomarkers is measured. In one embodiment, the level of at least 9 biomarkers is measured. In one embodiment, the level of at least 10 biomarkers is measured. In one embodiment, the level of at least 11 biomarkers is measured. In one embodiment, the level of at least 12 biomarkers is measured. In one embodiment, the level of at least 2 biomarkers up to all of the biomarkers of the disclosure are measured or any number between 2 and 30.

In some embodiments, the one or more, preferably two or more, biomarkers comprises or is IL-8. In some embodiments, the one or more, preferably two or more, biomarkers comprises or is IL-6. In some embodiments, the one or more, preferably two or more, biomarkers comprises or is sTNFR1. In some embodiments, the one or more, preferably two or more, biomarkers comprises or is sTREM1. In some embodiments, the one or more, preferably two or more, biomarkers comprises or is IL-10. In some embodiments, the one or more, preferably two or more, biomarkers comprises or is IL-1RA. In some embodiments, the one or more, preferably two or more, biomarkers comprises or is IL-2. In some embodiments, the two or more biomarkers comprises or is IL-4. In some embodiments, the one or more, preferably two or more, biomarkers comprises or is IL-7. In some embodiments, the one or more, preferably two or more, biomarkers comprises or is IL-9. In some embodiments, the one or more, preferably two or more, biomarkers comprises or is IL-13. In some embodiments, the one or more, preferably two or more, biomarkers comprises or is IL-17. In some embodiments, the one or more, preferably two or more, biomarkers comprises or is IFN-g. In some embodiments, the one or more, preferably two or more, biomarkers comprises or is IP-10. In some embodiments, the one or more, preferably two or more, biomarkers comprises or is MCP-1. In some embodiments, the one or more, preferably two or more, biomarkers comprises or is G-CSF. In some embodiments, the one or more, preferably two or more, biomarkers comprises or is GM-CSF. In some embodiments, the one or more, preferably two or more, biomarkers comprises or is FGF-basic. In some embodiments, the one or more, preferably two or more, biomarkers comprises or is SCGF-β. In some embodiments, the one or more, preferably two or more, biomarkers comprises or is GRO-a. In some embodiments, the one or more, preferably two or more, comprises or is MIP1-α. In some embodiments, the one or more, preferably two or more, biomarkers comprises or is MIP1-β. In some embodiments, the one or more, preferably two or more, biomarkers comprises or is CK-18. In some embodiments, the one or more, preferably two or more, biomarkers comprises or is PDGF-bb. In some embodiments, the one or more, preferably two or more, biomarkers comprises or is caspase 3. In some embodiments the one or more, preferably two or more, biomarkers comprises or is HMGB-1. In some embodiments, the one or more, preferably two or more, biomarkers comprises or is TNF α. In some embodiments, the one or more, preferably two or more, biomarkers comprises or is VEGF.

As demonstrated in the Examples, the modeling is slightly improved by including assessment of CRB-65. The CRB-65 score can be used as a further input in determining PO risk, for example whether a patient should be hospitalized or can be safely discharged.

The CRB-65 is a score from 0-4 and is calculated from: Age, Confusion, Respiratory Rate, and Blood Pressure. (example; https://medschool.co/tools/crb65). There are numerous references to the score in the literature (example: httbs://pubmed.ncbi.nlm.nih.gov/16789984/).

In some embodiments, one or more clinical scores is combined with the one or more biomarker levels, for example the panel biomarkers, and used as inputs. For example, the clinical score may (q)SOFA, SAPS, CURB-65 and/or APACHE.

Also provided is a method for reducing avoidable admissions. As demonstrated herein, methods described herein reduced avoidable admissions by 50% percent.

Accordingly in a further aspect, the disclosure provides a method for triaging a patient with a respiratory illness, the method comprising:
obtaining a sample obtained from the patient;
quantitatively measuring in the sample a polypeptide level of two or more biomarkers, the biomarkers comprising sTNFR1 and sTREM1, and optionally one or more of IL-6, IL-8 and IL-10, preferably wherein the two or more markers are sTNFR1, sTREM1, IL-6, IL-8 and IL-10; and
   i) comparing the level of the two or more biomarkers in the sample with a control or cut-off level, wherein the differential level is indicative of patient outcome risk; or
   ii) using the polypeptide level of several of the biomarkers in combination, as inputs for an algebraic calculation or machine learning model to determine whether the patient should be hospitalized or can be safely discharged.

Different combinations of markers can be used in any of the methods described herein. In an embodiment, the one or more biomarkers comprise at least sTNFR1 and sTREM1 and optionally any one, two or three of IL-8, IL-6, or IL-10.

In one embodiment, the one or more biomarkers are sTNFR1 and sTREM1.

In another embodiment, the one or more biomarkers are sTNFR1 and sTREM1 and one or more of IL-6, IL-8 or IL-10.

In an embodiment, the one or more biomarkers are IL-6, IL-8, sTREM1 and sTNFR1.

In yet another embodiment, the one or more biomarkers are sTNFR1, sTREM1, IL-6, IL-8 and IL-10.

In some embodiments, two biomarkers selected from IL-8, IL-6, sTNFR1 and sTREM1 are assessed. In yet other embodiments, three biomarkers selected from IL-8, IL-6, sTNFR1 and sTREM1 are assessed. It yet further embodiments, each of IL-8, IL-6, sTNFR1 and sTREM1 are assessed.

In some embodiments, the level of IL-6, CXCL8, IL-10, IL-1B, sTREM1, and sTNFR1 are measured.

The biomarkers levels may exclude in some embodiments, for example combinations consisting of only IL-2, IL-7, IL-10, G-CSF, IP10, MCP1, MIP1 α and/or TNF-α.

In some embodiments, the method includes first collecting a patient sample for example by venous puncture.

In an embodiment, the polypeptide level is indicative that the patient has an increased risk of poor PO, is an increase of at least 1.2×, 1.3×, 1.4×, 1.5×, 1.6×, 1.7×, 1.8×, 1.9×, 2×, 3×, 4×, 5×, 6×, 7×, 8×, 9× or 10× and/or up to 5×, up to 8× or up to 10×, optionally any value therebetween 2× and 10×, compared to control. For example the poor PO can be a requirement for ICU admission. In an embodiment, the polypeptide level is indicative that patient has an increased risk of poor PO is an increase of any value therebetween 2× and 5×, compared to control, e.g. wherein the poor PO is a requirement for ICU admission.

In an embodiment, the method, kit or immunoassay comprises a minimum lower limit of detection (LLOD) for each of the one or more biomarkers. In an embodiment, the LLOD is at least 1 pg/mL for IL-6, IL-8, and/or IL-10 and at least 15 pg/mL for sTNFR1 and/or sTREM1.

ELLA by Protein Simple (https://www.proteinsimple.com/ella.html) can be used to measure the levels of the one or more biomarkers, optionally the panel biomarkers. For example, in the ELLA system IL-6, IL-10, and IL-8 LODs are 0.1 pg/mL, sTREM-1 is 0.73 pg/mL, and sTNFR1 is 0.1 pg/mL with this assay. Other systems such as RALI-DX by SQI Diagnostics Systems can also be used.

Other systems can also be used. For example, a system with a LLOD for IL-6 that is at least 21 pg/mL, for IL-8 that is at least 27 pg/mL, for IL-10 that is at least 7 pg/mL, for sTNFR1 that is at least 17 pg/mL, and/or for sTREM1 that is at least 44 pg/mL can also be used.

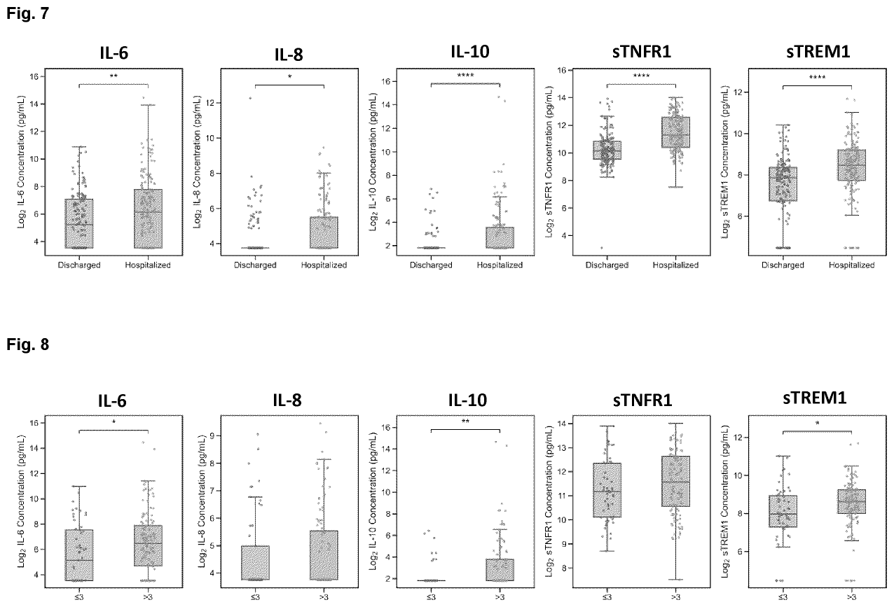

Example 3 provides an example of the median plasma levels of the Panel biomarkers in healthy controls. The median plasma levels of Panel biomarkers in healthy controls were below detection limits for IL-6, IL-8, and IL-10, and 574 pg/mL and 221 pg/mL for sTNFR1 and sTREM1. As shown in Table 2 and FIG. 7, all of the Panel biomarkers were significantly elevated in the plasma of patients that were hospitalized with respiratory illness compared to healthy controls as well as those that were discharged home. For example, the lower limit of detection of IL-6 using the 5-plex assay described in Example 3 is 21 pg/mL and the median level in patients that were hospitalized is 70 pg/mL or 3× the level of detection. Accordingly, in one embodiment, the level of IL-6 indicative of poor PO, optionally requiring hospitalization, is at or at least at 8×, 9×, 10×, 11×, 12×, 13× or 13.7× compared to a control wherein the control is comprised of healthy subjects (e.g. not experiencing a respiratory illness). If the control is patients with a non-severe respiratory illness not requiring hospitalization, an increase of or greater than 1.8×, 1.9× or 2× is indicative of poor PO, optionally requiring hospitalization. Similarly, a level about or less than, for example, 7× a control wherein the control is comprised of healthy subjects (e.g. not experiencing a respiratory illness) or about or less than 1.6× or 1.4× a control wherein the control is patients with a non-severe respiratory illness not requiring hospitalization.

The level of detection of IL-8 in the 5-plex assay described in Example 3 is 27 pg/mL and the interquartile level in patients that were hospitalized is 0-45 pg/ml. The interquartile level in discharged patients as well as healthy controls is below the level of detection. Accordingly, an IL-8 level at or greater than for example 30 pg/mL or 35 pg/mL, is indicative of poor PO, optionally requiring hospitalization. Similarly a level about or less than, for example, 30 pg/mL or 27 pg/mL is indicative of good PO, optionally safe discharge.

The level of detection of IL-10 in the 5 plex assay described in Example 3 is 7 pg/mL and the interquartile level in patients that were hospitalized is 0-12 pg/ml. The interquartile level in discharged patients as well as healthy controls is below the level of detection. Accordingly, an IL-10 level at or greater than for example 9 pg/mL or 10 pg/mL, is indicative of poor PO, optionally requiring hospitalization. Similarly a level about or less than, for example, 8 pg/mL or 7 pg/mL is indicative of good PO, optionally safe discharge.

Further as demonstrated the median level of sTNFR1 in healthy controls was 574 pg/mL whereas the median level in patients discharged was 1134 pg/mL and in patients hospitalized was 2507 pg/mL. Accordingly, in one embodiment, the level of sTNFR1 indicative of poor PO, optionally requiring hospitalization, is at or at least at 2.5×, 3×, 3.3×, 3.5×, 4× or 5× compared to a control wherein the control is comprised of healthy subjects (e.g. not experiencing a respiratory illness). If the control is patients with a non-severe respiratory illness not requiring hospitalization, an increase of or greater than 1.8×, 1.9×, 2× or 2.2× is indicative of poor PO, optionally requiring hospitalization. Similarly, a level about or less than for example 2× a control wherein the control is comprised of healthy subjects (e.g. not experiencing a respiratory illness) or about or less than 1.6× or 1.4× a control wherein the control is patients with a non-severe respiratory illness not requiring hospitalization.

Further as demonstrated the median level of sTREM1 in health controls was 221 pg/mL whereas the median level in patients discharged was 231 pg/mL and in patients hospitalized was 352 pg/mL. Accordingly, in one embodiment, the level of sTREM1 indicative of poor PO, optionally requiring hospitalization, is at or at least at 1.5×, 1.6×, 1.7×, 1.8×, 1.9× or 2× compared to a control wherein the control is comprised of healthy subjects (e.g. not experiencing a respiratory illness). If the control is patients with a non-severe respiratory illness not requiring hospitalization, an increase of or greater than of at least or about 1.3×, 1.4× or 1.5× is indicative of poor PO, optionally requiring hospitalization. Similarly, a level about or less than for example 1.5× a control wherein the control is comprised of healthy subjects (e.g. not experiencing a respiratory illness) or about or less than 1.2× a control wherein the control is patients with a non-severe respiratory illness not requiring hospitalization.

As indicated in the Examples, every 1 ng/mL increase in sTNFR1 and sTREM1 increased the risk of hospitalization by 1.3 and 8 fold respectively (Table 6). The median IL-6 values measured in the ED of patients that were at risk of intubation and mechanical ventilation were ˜4× higher than those patients not at risk (Table 10). A similar increase was observed for sTNFR1 (˜3×) and sTREM1 (˜3×) (Table 10).

As mentioned the methods can be used to predict patients at risk of intubation and mechanical ventilation, mechanical ventilation (optionally invasive or non-invasive) as well as ICU treatment.

In one embodiment, the concentration is the log 2 concentration.

A number of patient parameters can also be considered and/or combined with the biomarker level(s) for predicting patient outcome such as likelihood of ICU admission.

Also provided are uses of the methods described herein for screening or stratifying patients as less or more likely to require hospitalization, mechanical ventilation and/or ICU treatment. The methods are useful for example for reducing avoidable admissions.

The levels of the polypeptide biomarkers can be measured using assays, kits and platforms for measuring polypeptide levels. For example, the methods can include immunoassays such as ELISA and multiplex assays including Luminex® based assays, flow cytometry, Western blots, and immunoprecipitation followed by SDS-PAGE immunocytochemistry. Protein microarrays are also useful. Immunoassays and kits described herein can also be used.

Depending on the assay, the method can comprising contacting the sample with one or more detection agents that directly or indirectly produce a detectable signal, and measuring the signal.

In an embodiment, the level one or more polypeptide biomarkers described herein is detected or determined by immunohistochemistry (IHC), Luminex® based assays, Western blots, ELISA, immunofluorescence, radioimmunoassay, dot blotting, FACS, protein microarray, immunoprecipitation followed by SDS-PAGE, immunocytochemistry, Simple Plex assay or Mass Spectrometry. ELLA and RALI-Dx platforms can for example be used.

In an embodiment, the levels of the one or more, preferably two or more, of the polypeptide biomarkers described herein are detected for example using a Luminex® assay.

An at least 1.2× or 1.2 fold difference means, for example, that the level of the biomarker in the sample is at least 120% the level in a control comparator sample or derived value.

In an embodiment, the method involves comparing to a cut-off. For example each marker will have a different cut-off depending on statistical calculations and/or desired test sensitivity and/or specificity. Where more than one biomarker is assessed, a composite score can be determined.

The biomarker levels can, for example, be measured using various immunological and/or proteomic assays. For example, the polypeptide level of a biomarker of the disclosure can be measured using an ELISA).

The algebraic calculation and/or machine learning model may be a component of the device used to measure the level of the one of or more biomarkers. For example a device with a CPU may be programmed. In other embodiments, the algebraic calculation and or model may part of an imagine processing unit, optionally in the form of an “app”, for example comprises on a phone, tablet, computer or other similar type of equipment. As described in Example 4, the levels of the one or more biomarkers could be provided to the an urgent care or medical staff, the levels of the one or more biomarkers can be used as inputs by the urgent care or medical staff to determine patient outcome risk e.g. requirement for hospitalization, ICU and/or increased likelihood of death.

Another aspect of the disclosure includes computer implemented systems and processes arranged to carry out the aforementioned methods of predicting patient outcomes.

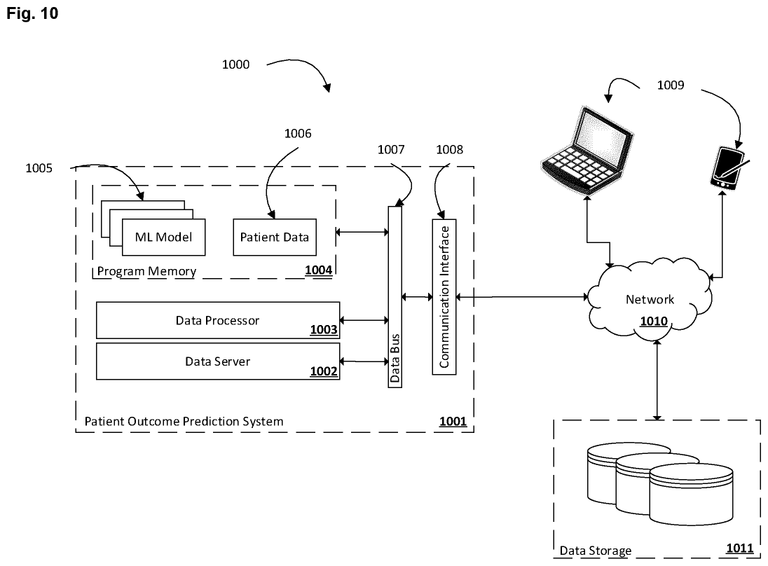

FIG. 10 is a schematic diagram of an example of a computing environment 1000 including a system in accordance with the present disclosure. The computing environment 1000 includes a patient outcome prediction system 1001 connected, via a network 1010, to other computing elements. Network 1010 may be a data communications network such as the Internet, and communication thereto/therefrom can be provided over a wired connection and/or a wireless connection (e.g. WiFi, WiMAX, cellular, etc.). The computing environment 1000 may also comprise communication devices 1009 configured to implement User Interfaces (UIs) for allowing users to interact with the patient outcome prediction system in such a way as to provide inputs thereto and receive outputs therefrom. In some embodiments, the computing environment 1000 includes data storage means used for storing development and training data for machine learning (ML) models 1005.

In some embodiments, the patient outcome prediction system 1001 shown in FIG. 10 is implemented in a single location. In other embodiments however, the patient outcome prediction system 1001 is distributed across a range of networked computing devices located in different physical locations. For example, the program memory 1004 containing ML models 1005 can form part of a cloud computing system.

In some embodiments, the patient outcome prediction system 1001 can be implemented within a larger software and/or hardware platform. For example, the patient outcome prediction system 1001 could be implemented within a platform that analyzes proteins. Similarly, the data server 1002 or servers used by the system to provide information to users via communication devices 1009 may also be located in different locations.

In some embodiments, the patient outcome prediction system 1001 includes one or more communication interfaces 1008 that may communicate via a wired connection or wireless connection to a network 1010 such as, for example, a Local Area Network (LAN) and/or the Internet. The various elements of patient outcome prediction system 1001 may be interconnected by a data bus 1007.

The patient outcome prediction system 1001 may also include a data processor 1003 and program memory 1004. As shown in FIG. 10, the patient outcome prediction system 1001 is connected to a data storage device 1011 via network 1010. In other embodiments however, the data storage device 1011 may form part of the patient outcome prediction system 1001. Data processor 1003 may comprise one or more processors for performing processing operations that implement functionality of the various methods described herein. Data processor 1003 may be a general-purpose processor executing program code stored in program memory.

Program memory 1004 comprises one or more memories for storing program code executed by data processor 1003, patient data 1006 used during operation of data processor 1003, as well as one or more ML models 1005. Program memory 1004 may be a semiconductor medium (including, e.g., a solid-state memory), a magnetic storage medium, an optical storage medium, and/or any other suitable type of memory.

In some embodiments, two or more elements of data processor 1003 may be implemented by devices that are physically distinct from one another and may be connected to one another via a bus (e.g., one or more electrical conductors or any other suitable bus) or via a communication link which may be wired. As will be appreciated by the skilled reader, the hardware components of the patient outcome prediction system 1001 may be implemented in any suitable way in order to implement the methods disclosed herein.

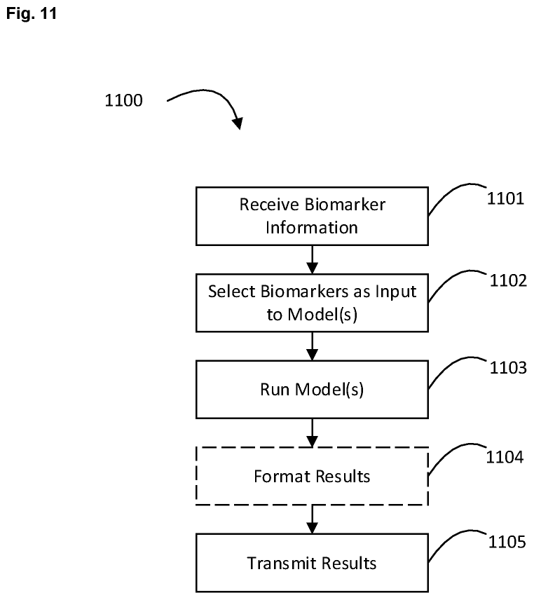

In some embodiments, the patient outcome prediction system 1001 shown in FIG. 10 is configured to perform the computer implemented method 1100 of FIG. 11. In particular, at step 1101 the patient outcome prediction system 1001 obtains biomarker information representing the biomarker polypeptide level values of a patient's plasma sample. In some embodiments, the biomarker information may be received from communication device 1009 or from any other suitable device. In other embodiments, the biomarker information may be created by the platform in which the patient outcome prediction system 1001 is implemented (as described herein) by measuring polypeptide levels of patient plasma samples.

At step 1102, a group of biomarkers are selected as inputs to the ML models 1005 from the biomarker information received at step 1101, as described elsewhere herein.

At step 1103, the ML models 1005 are run using the biomarkers selected at step 1102 as inputs. In some embodiments, the outputs of the ML models 1005 may be formatted at step 1104. Finally, at step 1105, the unformatted or formatted results are transmitted to a communication device 1009 for visualization by a user. In other embodiments, the results are transmitted to a display of the computing device upon which the patient outcome prediction system is implemented.

The ML models 1005 include at least one model trained using patient data. In one exemplary embodiment, ML models 1005 may be developed using a known software library. A non-limiting example of such a software library is the known Extreme Gradient Boosting™ (XGBoost™) software library, which provides a gradient boosting framework for solving regression and classification problems.

In one exemplary embodiment, three ML models are constructed, namely a “Panel” model, comprised of biomarkers IL-6, IL-8, IL-10, sTNFR1, and sTREM1, a “Panel+CRB-65” model, comprised of biomarkers IL-6, IL-8, IL-10, sTNFR1, sTREM1, and CRB-65, and a “Standard Model” model, based on biomarker CRB-65 alone. In this embodiment, each model is designed to predict hospitalization vs. safe discharge using the clinically adjudicated ETC-19 dataset, as described in more detail herein. The ETC-19 cohort is randomly partitioned 80:20 with 80% of data being used for development of the model and a 20% hold-out test set. 5-fold cross validation is then carried out ten times in the development set. The model is then applied to the ETC-19 test set and the Italian Cohort (as described in more detail herein) for validation. Model performances are then assessed using the area under the receiver operating characteristic curve (AUROC) with the null hypothesis that the AUROC is 50%. Model performance can be reported as mean AUROC with standard deviation where appropriate.

The predicted probabilities for each patient derived from the Panel+CRB-65 model can be used for post-hoc model analysis. The number of patients that developed severe illness (ARDS or bacteremia, required care in an ICU, respiratory support using mechanical ventilation, or died during the 28-day follow-up) are assessed for correctly predicting the need for hospitalization, based on the emergency department blood sample (as described in more detail herein). The Panel+CRB-65 model can be evaluated on the entire COVID-19 positive cohort for both model performance (AUROC) and correctly identifying patients at risk of severe illness. The relative feature importance of the biomarkers in the PANEL model can be given by their Shapley Additive Explanations (SHAP) values. As demonstrated in the Examples, the SHAP values identified were IL-6:0.01099, IL-8:0.015074, IL-10:0.04633, sTNFR1:0.298175 and sTREM1:0.162913.

While the use of decision trees is particularly advantageous to the applications described herein, the ML models 1005 of the patient outcome prediction system 1001 described herein may instead of (or together with) use other ML models including, but not limited to, linear regression models, logistic regression models, linear discriminant analysis models, naïve Bayes classifiers, K-nearest neighbors classifiers, learning vector quantization models, support vector machines, bagging and random forest models and deep neural networks.

A person of skill in the art will readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The program storage devices may be, e.g., digital memories, magnetic storage media such as a magnetic disks and magnetic tapes, hard drives, or optically readable digital data storage media. The embodiments are also intended to cover computers programmed to perform said steps of the above-described methods.

In some embodiments, the methods and systems described herein can be deployed using various platform as a service (PaaS) products such as, but not limited to, Docker™, provided by Docker, Inc.

The description and drawings merely illustrate the principles of the invention. It will thus be appreciated that those skilled in the art will be able to devise various arrangements that, although not explicitly described or shown herein, embody the principles of the invention and are included within the scope of the appended claims. Furthermore, all examples recited herein are principally intended expressly to be only for pedagogical purposes to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention, as well as specific examples thereof, are intended to encompass equivalents thereof.

The functions of the various elements shown in the Figures, including any functional blocks labelled as “processors”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. Moreover, explicit use of the term “processor” should not be construed to refer exclusively to hardware capable of executing software, and may implicitly include, without limitation, digital signal processor (DSP) hardware, network processor, application specific integrated circuit (ASIC), field programmable gate array (FPGA), read only memory (ROM) for storing software, random access memory (RAM), and non-volatile storage. Other hardware, conventional and/or custom, may also be included.

It should be appreciated by those skilled in the art that any block diagrams herein represent conceptual views of illustrative circuitry embodying the principles of the invention. Similarly, it will be appreciated that any flow charts, flow diagrams, state transition diagrams, pseudo code, and the like represent various processes which may be substantially represented in computer readable medium and so executed by a computer or processor, whether or not such computer or processor is explicitly shown.

### III. Immunoassays and Kits

An aspect of the disclosure also includes kits containing antibodies for the detection of the biomarkers of the disclosure that are used to measure the biomarker levels, i.e. polypeptide levels.

In an embodiment the kit comprises an immunoassay for one or more of biomarkers of the disclosure. Each kit or immunoassay comprises at least one detection antibody specific for a biomarker of the disclosure. For example, the antibody may be in the form of antibody coupled beads such as antibody coupled magnetic beads, or labelled antibodies, optionally comprised in a cartridge.

The cartridge may comprise a sample holding chamber for receiving and retaining the sample, a first conduit connected to said sample holding chamber, at least one biomarker sensor, and preferably two or more, optionally 5 biomarker sensors, each sensor comprising a biomarker responsive surface, wherein said surface is in the first conduit; a second conduit for retaining fluid, wherein said second conduit is fluidly connected to said first conduit; a mechanism for example comprising a pump, for displacing the sample from the holding chamber to the first conduit and for displacing the fluid from the second conduit into the first conduit; and an immunoassay composition for detecting each biomarker. The immunoassay composition comprises one or more detection antibodies for each biomarker. The cartridge can comprise a plurality of such configurations for measuring the level in a plurality of samples. The detection antibody can be labelled or a secondary labelled antibody can be utilized.

The immunoassay can, for example, be in the form of a plate such as a 96-well plate comprising one or more antibodies fixed thereto or for fixing thereto or a cartridge comprising one or more antibodies.

In an embodiment, the kit further comprises one or more of a plate such as a 96-well plate comprising one or more antibodies fixed thereto or for fixing thereto, a cartridge comprising one or more antibodies, standards, assay buffer, wash buffer, sample diluent, standard diluent, detection antibody diluent, streptavidin-PE, a filter plate and sealing tape.

In an embodiment the kit or immunoassay comprises detection antibodies or assay(s) (e.g. ELISA plate) for detecting two or more biomarkers of the disclosure e.g. two or more of IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α, VEGF, sTNFR1 and sTREM1. In some embodiments, the two or more biomarkers of the disclosure comprise sTREM1 and sTNFR1. In other embodiments, the two or more biomarkers of the disclosure comprise sTREM1 and sTNFR1 and one, two or three of IL-6, IL-8, or IL-10. The two or more biomarkers can be any combination of the biomarkers of the disclosure.

In an embodiment, the kit or immunoassay comprises detection antibodies or assay(s) for detecting IL-6, IL-8, IL-10, sTREM1 and sTNFR1.

In some embodiment, the kit comprises components of a multiplex assay or the immunoassay is a multiplex assay. For example, the kit can comprise or the immunoassay can be a plate, such as a 96 well plate or a cartridge that comprises a plurality of wells, chambers or conduits, wherein a subset thereof comprise one or more standards optionally a standard based on for example values in healthy controls, values in known outcome patients (e.g. comprising a respiratory illness where patient can be safely discharged) a first threshold (e.g. low control) and a second threshold (e.g. high control). The subset may also comprise a known level of each biomarker to be tested in the form of a standard curve. Alternatively, the kit can comprise one or more standards for preparing a standard curve.

The detection antibody, secondary antibody etc in the kit, cartridge, bead, plate and/or assay can be a Fab fragment. The antibody can be covalently attached to the cartridge, bead, plate or other assay component.

The beads can for example comprise latex beads, polystyrene beads, or acrylic beads or combinations thereof. Examples used in the art include Bio-Plex by BioRad powered by Luminex xMAP; or MagFlex® by Luminex, BD™ CBA Flex Set.

The label conjugated to the detection antibodies can be any signal generating element, for example a radiolabel, metal particle, fluorescent dye, chromogenic dye, labeled protein, enzyme, or combinations thereof. The enzyme can for example comprise peroxidase, glucose oxidase, phenol oxidase, β-galactosidase, alkaline phosphatase, or combinations thereof.

In an embodiment, the kit or immunoassay is for use in a method described herein.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

Blood samples will be collected from patients with a respiratory illness. The blood samples will be processed and analyzed for one or more of the polypeptide levels of IL-6, CXCL8, IL-10, IL-1β, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, ET-1, CK-18, PDGF-bb, caspase 3, HMGB-1, TNF α, VEGF, sTNFR1 and sTREM1, optionally IL-6, CXCL8, IL-10, IL-1B, sTREM1, sTNFR1, and ET-1 or IL-6, IL-8, sTNFR-1, and sTREM1.

The polypeptide levels (either absolute concentration, or relative concentration (e.g. compared to a health control) are included in a mathematical calculation that may include other known clinical variables (e.g. underlying disease, age). The output of this calculation provides a predictive score of whether or not the patient is likely to require hospitalization, mechanical ventilation or ICU stay. At that point the patient outcome risk can be assessed and treatment or release can be assessed.

### Methods

Patient Selection: Patients arriving at Emergency Departments presenting symptoms of fever and/or onset of cough or difficulty breathing. Subject inclusion criteria: Fever and/or Onset of cough OR difficulty breathing (symptoms of respiratory infection)

Blood Sample. A Nurse will collect a blood sample in a coagulated vacutainer. Once subjects are diagnosed with respiratory distress (SARS-nCoV-2) by nasopharyngeal swab, their blood sample will be used for the assay. The blood sample will be centrifuged at 3,100 rpm for 10 minutes to fractionate the blood, and the plasma will be collected

Biomarker Quantification: Blood samples are optionally processed to provide serum or plasma and diluted as per manufacturer's instructions in calibrator diluent. A set of standards for the generation of a standard curve is prepared concurrently. Each plate was prepared according to the manufacturer's protocol. Each plate is then run and read on the Simple Plex System (Protein Simple), which is set up and calibrated as per the manufacturer's instructions.

Statistical Analysis: Regression analysis is carried out using Prism 7 (GraphPad), SPSS Statistics (IBM), or R software environment. Development of the predictive models is performed using logistic regression analysis on the measured biomarkers and carried out in all combinations of measured markers. Cross-validation of each model is carried out using 100 rounds of 10-fold cross-validation with stratification. For all statistical calculations, a p-value of less than 0.05 is considered statistically significant.

Previous transplantation studies, have identified markers in isolated lung perfusate samples (using EVLP) that showed a strong relationship between the expression of the markers and outcome (e.g. such as poor lung function). Poor lung function can lead to an organ being declined for transplantation or, if transplanted, a poor patient outcome (such as long ICU stay).

### Example 2

Various biomarkers were assessed in preliminary assessments in patients with respiratory illness. Biomarkers sTNFR1 and sTREM1 levels consistently associated with severity of respiratory disease. ET-1 and IL-1β were inconsistent and/or not associated with patient outcome.

### Example 3

Biomarkers for Detecting of Outcome/Risk of Patients with a Respiratory Illness

Based on preliminary assessments, a panel of biomarkers IL-6, IL-8, IL-10, sTNFR1 and sTREM1 for further assessed for triaging patients presenting at an emergency department with respiratory illness.

### Materials and Methods

Patient Population and Data Source: A prospective observational multicenter study comprising three cohorts. ETC-19 Cohort: Patients presenting to a UHN (Toronto, ON, CAN) emergency department (ED) with symptoms of respiratory illness were approached to participate in the ETC-19 study. An additional set of patients that tested positive for COVID-19 and had blood samples drawn at UHN were included. Italian Cohort: Patients presenting to the ED of Citta della Salute e della Scienza di Torino Hospital-Molinette Site (Turin, ITA) were approached to participate in this study. Both cohorts included COVID-19 patients. Controls: Healthy staff at Citta della Salute e della Scienza di Torino Hospital-Molinette Site (Turin, ITA) were approached to provide blood samples for the control arm of this study.

Participant Details: A whole blood sample was collected via venipuncture from participating patients as part of routine blood work during ED evaluations. The COVID-19 status of each patient was confirmed using a nasopharyngeal (NP) reverse transcription (RT) PCR swab for the presence of SARS-CoV-2 or through a previously confirmed RT PCR test result. Basic demographic details were collected from each patient alongside standard vital signs assessments and hospitalization metrics. All patients were followed for 28-days post ED visit via medical records and/or phone call. Patients who withdrew from the study at any time were excluded from analysis. All studies were reviewed and approved by the Research Ethics board of each institution.

Clinical Adjudication: All cases in the ETC-19 study were independently adjudicated by two clinicians for the following fields: avoidable admission, COVID-19 disease severity and pneumonia, acute respiratory distress syndrome (ARDS), bacteremia, community acquired pneumonia, ventilator-associated pneumonia, and cause-of-death. Standardized definitions were used (Clinical Management of COVID 19 Interim Guidance WHO; WHO/2019-nCoV/clinical/2020.5; CDC/NHSN surveillance definitions for specific type of infections January 2). Avoidable admissions were defined as patients who were discharged from hospital within 72-hours and did not require significant medical intervention. Any discrepancies between clinicians were resolved via paired-review.

Biomarker Panel: A 5-plex protein-based assay (Panel) was developed and included the following markers: IL-6, IL-8, IL-10, sTNFR1, sTREM1. Whole blood samples collected in the emergency department were sent to the clinical lab for plasma processing. Samples were stored at 4° C. for immediate testing or at −80° C. for batched testing. Samples were diluted 1:1 in assay diluent. 60 uL of diluted plasma was loaded on a custom 96-well microtitre plate that contained a standard curve and a high and low positive control derived from the reference standard for each biomarker. The World Health Organization (WHO) reference standard was used for IL-6, IL-8, and IL-10, and a Quantikine ELISA standard (R&D Systems, MN, USA) was used for sTNFR1 and sTREM1. Cytokine concentrations for each biomarker were determined by quantitative immunofluorescence using the automated 60-minute sqidlite system (SQI Diagnostics, ON, CAN). Analytical validation of the biomarker panel has been completed according to standard guidelines ((e.g. CLSI (Clinical & Laboratory Standars Institute) Guidelines). The limit of detection for each biomarker was: IL-6 (21 pg/mL), IL-8 (27 pg/mL), IL-10 (7 pg/mL), sTNFR1 (17 pg/mL), sTREM1 (44 pg/mL).

Triage Model Development: The ED triage model was developed using the Extreme Gradient Boosting (XGBoost) machine learning algorithm. Three models were constructed: the ‘Panel’ model (comprised of IL-6, IL-8, IL-10, sTNFR1, and sTREM1), the ‘Panel+CRB-65’ model (comprised of IL-6, IL-8, IL-10, sTNFR1, sTREM1, and CRB-65), and the ‘Standard Model’ model (based on CRB-65 alone). Each model was designed to predict hospitalization (e.g. poor outcome) vs. safe discharge (e.g. good outcome) using the clinically adjudicated ETC-19 dataset. The ETC-19 cohort was randomly partitioned 80:20 with 80% of data being used for development and a 20% hold-out test set. 5-fold cross validation was carried out 10-times in the development set. The model was then applied to the ETC-19 test set and Italian cohort for validation. Model performance was assessed using the area under the receiver operating characteristic curve (AUROC) with the null hypothesis that the AUROC was 50%; model performance was reported as mean AUROC with standard deviation where appropriate.

Post-Hoc Model Assessment: The predicted probabilities for each patient derived from the Panel+CRB-65 model was used for post-hoc model analysis. Briefly, the number of patients that developed severe illness (ARDS or bacteremia, required care in an ICU, respiratory support using mechanical ventilation, or died during the 28-day follow-up) were assessed for correctly predicting the need for hospitalization, based on the ED blood sample. The Panel+CRB-65 model was evaluated on the entire COVID-19 positive cohort for both model performance (AUROC) and correctly identifying patients at risk of severe illness.

Study Outcomes: The primary outcome was to investigate the predictive accuracy of biomarkers measured at clinical presentation in the ER to determine: 1) Good prognosis (discharge home) vs. 2) High risk prognosis (requiring admission to hospital or ICU). Secondary outcome was to investigate the association between these severity markers and subsequent clinical outcome data in the ER to determine: 1) Good prognosis (discharge home) vs. 2) High risk prognosis (requiring admission to hospital or ICU).

Statistical Analysis: All analysis was conducted using Stata (StataCorp, TX, USA), GraphPad (GraphPad Software, CA, USA), SPSS Statistics (IBM Corp, NY, USA), or R statistics software. Descriptive statistics of patient enrollment characteristics were evaluated using Chi-squared, Fisher's exact, or Mann-Whitney U tests, as appropriate, to determine patient factors associated with clinical outcomes. For logarithmic graphs, protein concentrations below the lower limit of detection (LLOD), samples were assigned a value of 0.5*LLOD in the corresponding figures. Protein distributions followed a non-parametric distribution and were assessed using the Mann-Whitney U or Kruskal-Wallis tests, where appropriate. Multiple comparisons were made using Dunn's correction of the Kruskal-Wallis test. Correlation between protein concentrations and duration of hospitalization was evaluated using non-parametric statistics and reported as Kendall's Tau. The predictive ability of the individual biomarkers was assessed using odds ratios (OR) and AUROC, with the null hypothesis that the AUROC was 50%. A threshold of p<0.05 was considered significant in this study.

### Results

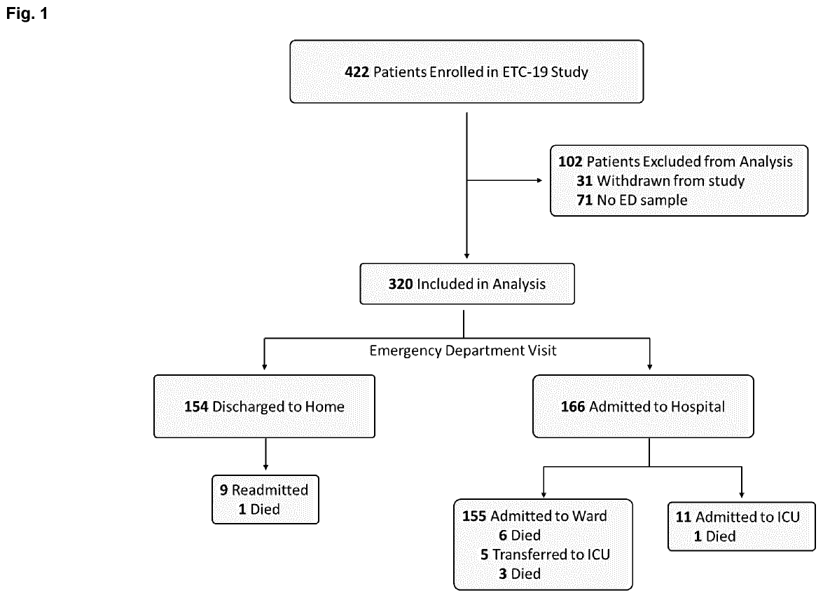

The ETC-19 cohort is representative of the ED population. During the study, there were n=320 patients that presented to a UHN ED with respiratory illness. The initial ED triage decision to hospitalize patients is shown in FIG. 1. Approximately 52% of patients were admitted to hospital following their ED visit (FIG. 1) and a total of 5% required care in an ICU during a four-week period (FIG. 1). Overall, the 28-day mortality rate was 3.4% (FIG. 1) in the ETC-19 cohort. The ED population was predominantly male (59%), Caucasian (59%), and presented with shortness of breath (dyspnea) (52%) (Table 1). Consistent with Provincial averages over the same period (mean: 2.2%, min.: 0.4%, max.: 9.0%, Public Health Ontario), the positivity rate of COVID-19 in the ETC-19 cohort was 6.9% (Table 1). Panel protein levels for the ETC-19 population are listed in Table 1.

Enrollment in Italy occurred during a peak period of COVID-19 (April-May 2020) and, as such, the positivity rate for COVID-19 was 43% in this cohort (Table 3). Similar to UHN, the cohort in Italy was predominantly Caucasian (75%) and presented with dyspnea (52%) (Table 3); however, the Italian cohort was predominantly female (58%) and older than ETC-19 (60 vs. 55 years old) (Tables 1 and 3). In the Italian cohort, 54% of patients were hospitalized and the incidence of ICU care (9.2%) and 28-day mortality (9.9%) were higher than that at UHN (Table 3).

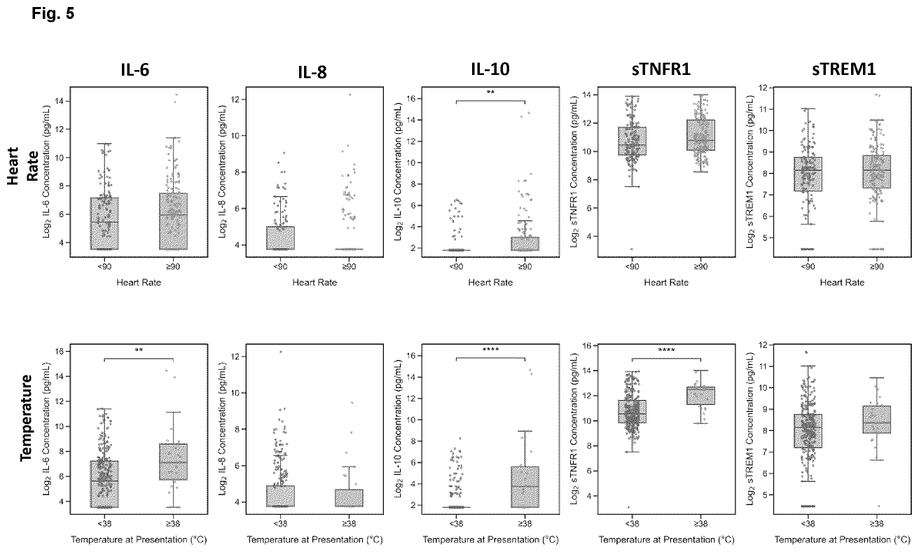

Panel biomarkers are associated with ED assessments: Levels of sTNFR1 and sTREM1 were significantly higher in males and increased with patient age (FIG. 51); there were no significant differences in Panel biomarkers based on patient ethnicity or BMI (FIG. 43 IL-6, IL-10, and sTNFR1 were significantly higher in patients that presented with fever (>38° C.) (FIG. 5), and there was a trend towards better oxygen saturation values with lower levels of these markers, although this did not reach statistical significance (FIG. 5). IL-10, sTNFR1, and sTREM1 levels were increased in patients with CRB-65 scores above 0 (FIG. 5).

Panel biomarkers are elevated in hospitalized patients: Table 2 shows that the majority of ED assessments (i.e., temperature, oxygen saturation, respiratory rate, blood pressure, etc.) are significantly associated with the decision to hospitalize patients following ED presentation; only BMI and COVID-19 status were not significantly associated with the decision to hospitalize (Table 2). Interestingly, male gender was significantly associated with the decision to hospitalize in the ETC-19 cohort (Table 2).

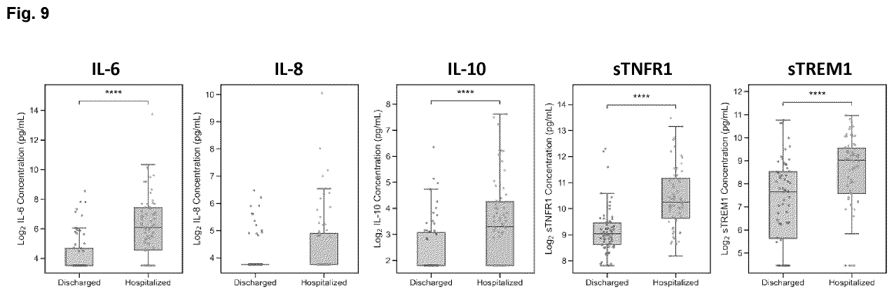

All Panel biomarkers (IL-6, IL-8, IL-10, sTNFR1, sTREM1) were significantly elevated in the plasma of patients that were hospitalized with respiratory illness (Table 2, FIG. 7). Median IL-6 and sTNFR1 plasma levels were approximately two-fold higher in hospitalized patients compared to those patients that were discharged for at-home recovery (Table 2, FIG. 7). Univariate logistic regression analysis of Panel biomarkers shows that each biomarker significantly predicts the decision to hospitalize (Table 5); sTNFR1 and sTREM1 were the strongest univariate predictors with AUROC values of 74% and 68% respectively (Table 5). Cut-offs based on Youden's J statistic for each biomarker are listed in Table 5; notably, for patients with undetectable levels of IL-10 in the blood (<7 pg/mL), the specificity was 100% for safe discharge home (Table 5). Furthermore, for every 1 ng/mL increase in sTNFR1 and sTREM1, the risk of hospitalization increases by 1.3- and 8.0-fold respectively (Table 6). Consistent with the ETC-19 cohort, elevated plasma levels of Panel biomarkers were also observed in patients that were hospitalized in Italy (FIG. 9).

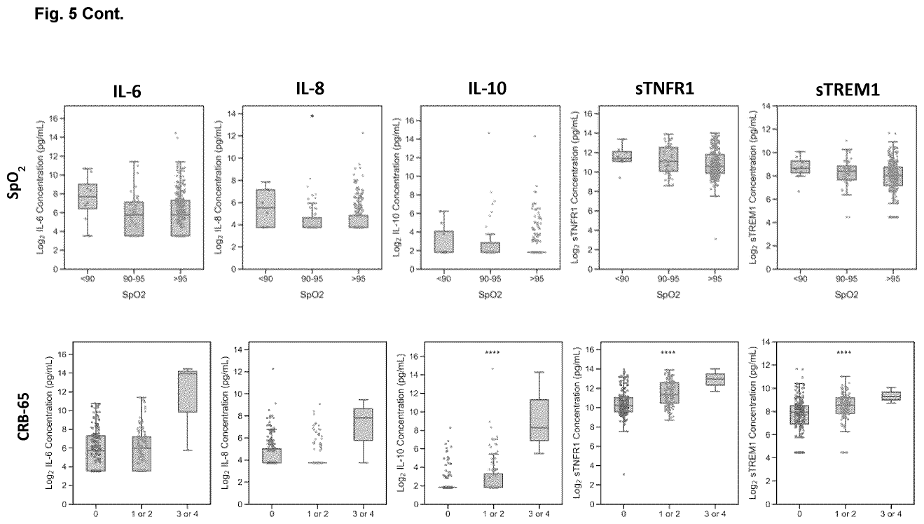

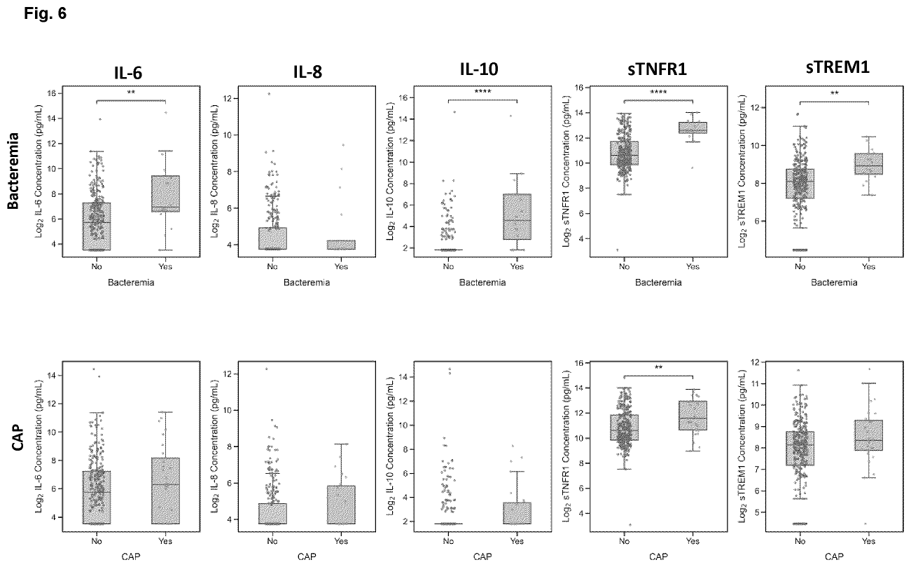

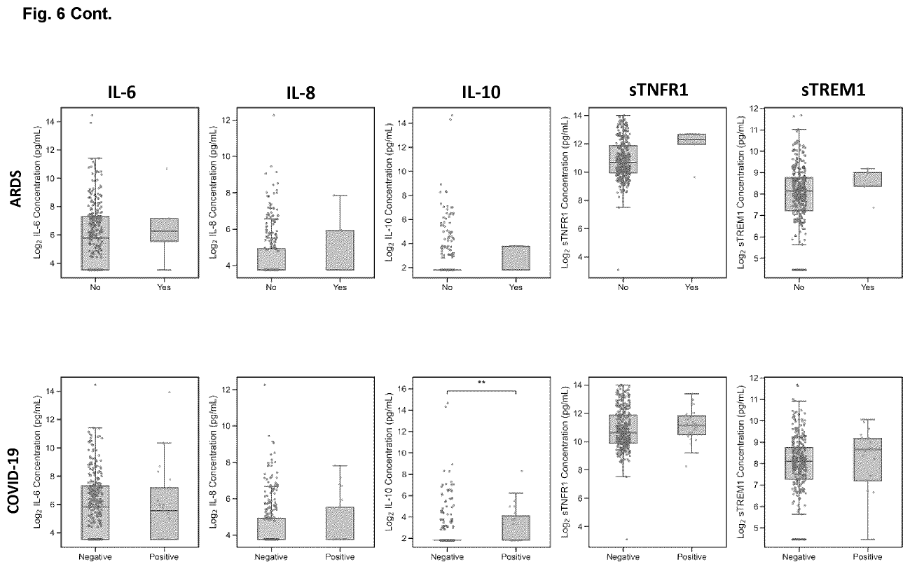

Panel biomarkers predict severity of respiratory illness: The plasma levels of IL-8 and sTNFR1 were significantly elevated in patients that died during the 28-day follow-up period (FIG. 2); the other Panel biomarkers were also elevated, but did not reach statistical significance (FIG. 2). Similarly, all Panel biomarkers were elevated in patients that required ICU care due to their respiratory illness with both IL-8 and IL-10 reaching statistical significance (FIG. 2). Only IL-10 showed a significant difference in patients that were diagnosed with COVID-19 (FIG. 6); however, the levels of all Panel biomarkers were significantly associated with the severity of COVID-19 illness (FIG. 2). Combinations of Panel biomarkers were significantly elevated in patients that developed bacteremia (FIG. 6), community acquired pneumonia (CAP) (FIG. 6), or COVID-19 pneumonia (FIG. 2). Although the incidence of ARDS was low (1.6%) in the ETC-19 cohort, Panel biomarkers were increased in patients with ARDS (FIG. S3). IL-6, IL-10, sTNFR1, and sTREM1 levels were significantly correlated with the duration of total hospitalization (Table 4); using a threshold of hospitalizations greater than 72-hours, IL-6, IL-10, and sTREM1 were significantly elevated in the plasma of these patients upon ED presentation (FIG. 8). In healthy patients, the median plasma levels of Panel biomarkers were below detection limits for IL-6, IL-8, and IL-10, and 574 pg/mL and 221 pg/mL for sTNFR1 and sTREM1, respectively (Table 9).

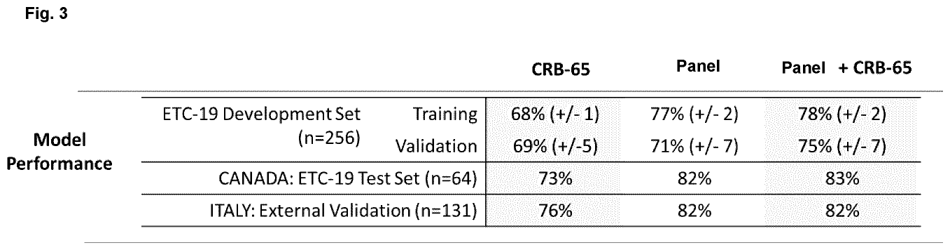

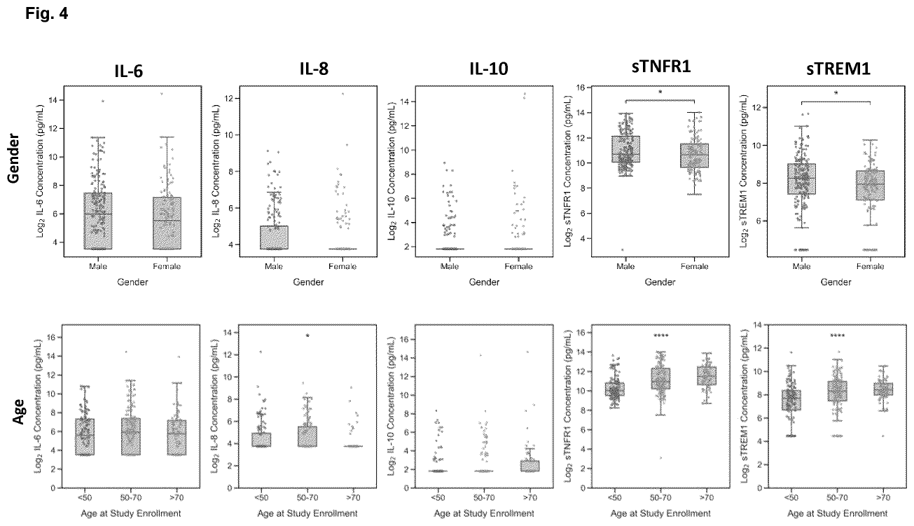

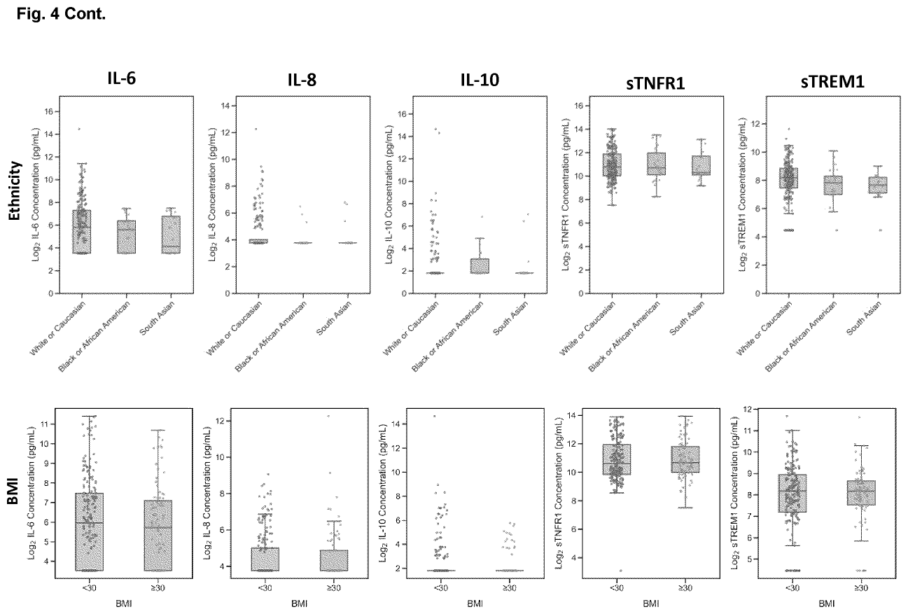

Panel biomarkers can be combined into a highly accurate triage model: Using the XGBoost algorithm, a machine learning tool, several models were developed to predict patients that required hospitalization for respiratory illness based on their Panel biomarker profile obtained in the ED. Using the clinically adjudicated ETC-19 cohort, patients were divided into two groups: patients that were “discharged safely” (discharged without the need for further care or an avoidable admission, n=166) versus those that “required hospitalization” (unavoidable admissions, n=154) (Table 11). Thus, the models were trained to predict those patients with respiratory illness for whom hospitalization was required. The ETC-19 cohort was randomly partitioned 80:20 for training and testing (n=256 and 64); the Italy cohort was used as additional, external validation (n=131). A model based entirely on Panel biomarkers (IL-6, IL-10, IL-8, sTNFR1, and sTREM1) had an AUROC of 77%, 71%, 82%, and 82% in the training, validation, test (ETC-19), and external validation (Italy) datasets (FIG. 3). For comparison, a model based on CRB-65 scores had a performance of 73% and 76% in the test (ETC-19) and external validation (Italy) datasets (FIG. 3), respectively. When CRB-65 scores were added to Panel, there was a slight improvement of the combined model (FIG. 3) compared to either model alone. Models that included Panel biomarkers showed that sTNFR1 and sTREM1 are important variables driving model predictions (Table 12).

The Panel+CRB-65 model correctly predicts hospitalization in patients with severe illness: A post-hoc analysis of important patient sub-populations was conducted using the results derived from the Panel+CRB-65 model. For patients with respiratory symptoms that died during the 28-day follow-up period, the model predicted hospitalization in 23 out of 24 patients (Table S5). Similarly, 87.5% of patients that eventually required ICU care and mechanical ventilation were categorized as patients requiring hospitalization using the Panel+CRB-65 model (Table S5) at ED presentation. A high level of accuracy was also observed for patients that required mechanical ventilation (Table 7). Post-hoc analysis of the accuracy of the Panel score to predict hospitalization for patients that had bacteremia was 100% (16/16) and, for patients that developed ARDS, the model accuracy was 100% (5/5). Importantly, post-hoc analysis of avoidable admissions in ETC-19 using Panel+CRB65 showed that the test would have correctly diverted ˜50% of these patients away from the hospital for safe at-home recovery. ˜57% of patients that were deemed to be avoidable admissions would have been diverted from the ED using the panel biomarkers alone. For COVID-19 patients, the Panel+CRB-65 model had an AUROC of 77% for the prediction of hospitalization (Table 8). In addition, the model correctly identified hospitalization for all patients that required invasive mechanical ventilation, 67% of patients that required non-invasive mechanical ventilation, 83% of patients that required ICU care, and 91% of patients that died (Table 8).

The Panel+CRB-65 model correctly predicts risk of intubation and mechanical ventilation in COVID-19 patients: Panel biomarkers were assessed to see if they were predictors of the risk of intubation and mechanical ventilation in COVID-19 patients. Univariate analysis of Panel biomarkers shows that each biomarker significantly predicts the risk of intubation with mechanical ventilation in COVID-19 positive patients (Table 10); sTNFR1, IL-6, and sTREM1 were the strongest univariate predictors with AUROC values of 88%, 81%, and 80% respectively (Table 10). The median IL-6 values measured in the ED of patients that were at risk of intubation and mechanical ventilation were ˜4× higher than those patients not at risk (Table 10). A similar increase was observed for sTNFR1 (˜3×) and sTREM1 (˜3×) (Table 10).

The importance of each biomarker in the Panel model was assessed. Table 12 shows the Shapley Additive Explanations (SHAP) values for the biomarkers in the ML model. Feature importance for the given biomarker is reported as SHAP value (mean, absolute value). The SHAP values show that sTRNFR1, followed by sTREM1, has the strongest contribution to the model.

### Example 4

A patient entering an urgent care setting presents with respiratory illness symptoms and is seen by a medical staff. A blood sample is drawn and the sample sent for processing, optionally in a patient identified vessel, and measuring one or more biomarkers. The sample may be sent to for processing and measuring to an outside facility or may be processed and/or measured internally. The medical staff or the urgent care centre receives optionally thru use of an app on a phone, tablet or other computer, a communication related to the patient. The communication may comprise levels of the one or more biomarkers or state the patient outcome, optionally whether the patient is safe to discharge, requires hospitalization, ICU and/or ventilation. If the levels of the one or more biomarkers is received, the medical staff or urgent care centre may use another app, computer etc, to determine patient outcome risk for example safe discharge or requirement for hospitalization.

### Tables

TABLE 1
Patient characteristics at ED baseline.

| | All Patients (n = 320) | |
|---|---|---|
| Mean Age (SD) - years | 55 | (18) |
| Male | 188/320 | (59%) |
| Race/ethnicity - White/Caucasian | 188/320 | (59%) |
| Race/ethnicity - Black/African | 21/320 | (6.6%) |
| American | | |
| Race/ethnicity - South Asian | 16/320 | (5.0%) |
| Mean BMI (SD) | 27.4 | (7.8) |
| COVID-19+ | 22/320 | (6.9%) |
| Fever | 79/320 | (26%) |
| Sore Throat | 62/320 | (20%) |
| Dyspnea | 162/320 | (52%) |
| Chest Pain | 90/320 | (30%) |
| Loss of Taste | 24/320 | (7.9%) |
| Loss of Smell | 19/320 | (6.3%) |
| Myalgia | 101/320 | (33%) |
| Fatigue | 183/320 | (60%) |
| IL-6 pg/mL (Median [IQR]) | 55 | [0-156] |
| IL-8 pg/mL (Median [IQR]) | 0 | [0-31] |
| IL-10 pg/mL (Median [IQR]) | 0 | [0-0] |
| sTNFR1 pg/mL (Median [IQR]) | 1629 | [976-3772] |
| sTREM1 pg/mL (Median [IQR]) | 286 | [155-438] |

TABLE 2
Summary of ED assessments in ETC-19 cohort by patient triage.

| | Discharged Home | | Hospitalized | | p-value |
|---|---|---|---|---|---|
| n | 154 | | 166 | | — |
| Male | 79 | (51%) | 109 | (66%) | 0.013 |
| Mean Age (SD) - years | 49 | (18) | 62 | (17) | <0.001 |
| Mean BMI (SD) | 26.6 | (6.7) | 28.2 | (8.7) | 0.082 |
| COVID-19 | 7 | (4.5%) | 15 | (9.0%) | 0.172 |
| Mean Temperature ° C. (SD) | 36.7 | (0.6) | 36.9 | (0.9) | 0.029 |
| Mean SpO₂ (SD) | 98 | (1) | 96 | (3) | <0.001 |
| Mean Respiratory Rate (SD) | 18 | (2) | 21 | (5) | <0.001 |
| Mean Heart Rate (SD) | 89 | (18) | 96 | (21) | 0.004 |
| Mean SBP (SD) | 135 | (22) | 128 | (25) | 0.007 |
| Mean DBP (SD) | 82 | (12) | 76 | (15) | <0.001 |
| Confusion | 2 | (1%) | 10 | (6%) | 0.049 |
| CRB-65 Score = 0 | 113 | (76%) | 65 | (41%) | <0.001 |
| CRB-65 Score > 0 | 36 | (24%) | 94 | (59%) | |
| IL-6 pg/mL (Median [IQR]) | 37 | [0-134] | 70 | [0-218] | 0.004 |
| IL-8 pg/mL (Median [IQ.R]) | 0 | [0-0] | 0 | [0-45] | 0.013 |
| IL-10 pg/mL (Median [IQR]) | 0 | [0-0] | 0 | [0-12] | <0.001 |
| sTNFR1 pg/mL (Median [IQR]) | 1134 | [751-1824] | 2507 | [1345-6053] | <0.001 |
| sTREM1 pg/mL (Median [IQR]) | 231 | [107-328] | 352 | [214-590] | <0.001 |

TABLE 3
Patient characteristics at ED baseline for the Italian cohort.

| | Italy (n = 131) | |
|---|---|---|
| Mean Age (SD) - years | 60 | (20) |
| Male | 54/130 | (42%) |
| Race/ethnicity - White/Caucasian | 42/56 | (75%) |
| Race/ethnicity - Black/African | 3/56 | (5.4%) |
| American | | |
| Race/ethnicity - Hispanic | 10/56 | (18%) |
| Mean BMI (SD) | — | |
| COVID-19+ | 56/131 | (43%) |
| Fever | 75/131 | (57%) |
| Sore Throat | 11/131 | (8.4%) |
| Dyspnea | 68/131 | (52%) |
| Chest Pain | 28/131 | (21%) |
| Loss of Taste | 23/131 | (18%) |
| Loss of Smell | 16/131 | (12%) |
| Myalgia | — | |
| Fatigue | — | |
| Hospitalized | 71/131 | (54%) |
| Required ICU Care | 12/131 | (9.2%) |
| 28-day Mortality | 13/131 | (9.9%) |
| IL-6 pg/mL (Median [IQR]) | 30 | [0-101] |
| IL-8 pg/mL (Median [IQR]) | 0 | [0-28] |
| IL-10 pg/mL (Median [IQR]) | 0 | [0-13] |
| sTNFR1 pg/mL (Median [IQR]) | 730 | [466, 1384] |
| sTREM1 pg/mL (Median [IQR]) | 304 | [97, 601] |

TABLE 4
Summary of Kendall's rank correlation of RALI-Dx
biomarkers with total hospital length of stay.

| | Correlation (tau) | p-value |
|---|---|---|
| IL-6 | 0.124 | 0.011 |
| IL-8 | 0.097 | 0.112 |
| IL-10 | 0.186 | 0.002 |
| sTNFR1 | 0.157 | 0.004 |
| sTREM1 | 0.167 | 0.002 |

TABLE 5
Univariate logistic regression results for RALI-Dx biomarkers
to predict hospitalization following ED presentation.

| | | | Cut-off | | |
|---|---|---|---|---|---|
| | AUROC | 95% CI | (pg/mL) | Sensitivity | Specificity |
| IL-6 | 59% | 53-65% | 52 | 60% | 56% |
| IL-8 | 56% | 51-61% | 699 | 100% | 1% |
| IL-10 | 60% | 56-65% | >7 | 0% | 100% |
| sTNFR1 | 74% | 69-80% | 2176 | 60% | 81% |
| sTREM1 | 68% | 62-74% | 370 | 51% | 80% |

TABLE 6
Summary of odds ratio for changes (pg/mL) in RALI-Dx biomarkers
to predict hospitalization following ED presentation.

| | | |
|---|---|---|
| Plasma Change | OR [95% CI] | p-value |

| | | | | | |
|---|---|---|---|---|---|
| IL-6 | 100 | pg/mL | 1.1 | [1.0-1.2] | 0.010 |
| IL-8 | 100 | pg/mL | 1.0 | [0.9-1.1] | 0.968 |
| IL-10 | 100 | pg/mL | 6.2 | [1.8-22.2] | 0.005 |
| sTNFR1 | 1,000 | pg/mL | 1.3 | [1.2-1.5] | <0.001 |
| sTREM1 | 1,000 | pg/mL | 8.0 | [3.3-19.3] | <0.001 |

TABLE 7
Post-hoc analysis of the RALI-DX + CRB-65 model to
predict hospitalization for patients with critical illness.

| | |
|---|---|
| ETC-19 | Italy |

| | | | | |
|---|---|---|---|---|
| 28-day Mortality | 100% | (11/11) | 92.3% | (12/13) |
| Mechanical Ventilation | 100% | (5/5) | 81.8% | (9/11) |
| (Any) | | | | |
| Invasive MV | 100% | (5/5) | 100% | (1/1) |

| | | | |
|---|---|---|---|
| Non-lnvasive MV | — | 80% | (8/10) |

| | | | | |
|---|---|---|---|---|
| ICU Care | 87.5% | (14/16) | 83.3% | (10/12) |

TABLE 8
Analysis of the RALI-DX + CRB-65 model to predict hospitalization
and critical illness for n = 103 C0VID-19+ patients.

| | COVID-19+ | |
|---|---|---|
| Model AUROC | 77% | |
| 28-day Mortality | 91% | (10/11) |
| Mechanical Ventilation | 80% | (8/10) |
| (Any) | | |
| Invasive MV | 100% | (4/4) |
| Non-Invasive MV | 67% | (4/6) |
| ICU Care | 83.3% | (10/12) |

TABLE 9
RALI-Dx biomarker levels measured
in n = 20 healthy control subjects.

| | Healthy Controls | |
|---|---|---|
| | pg/mL (Median [IQR]) | |
| IL-6 | 0 | [0-0] |
| IL-8 | 0 | [0-0] |
| IL-10 | 0 | [0-0] |
| sTNFR1 | 574 | [531-666] |
| sTREM1 | 221 | [133-296] |

TABLE 10
Univariate logistic regression results for RALI-Dx biomarkers to predict risk
of intubation and mechanical ventilation in n = 101 COVID-19+ patients.

| | Intubation and | | | | | |
|---|---|---|---|---|---|---|
| | MV Risk pg/mL | | Not at risk pg/mL | | | |
| | (Median [IQR]) | | (Median [IQR]) | | p-value | AUROC |
| IL-6 | 139 | (56-301) | 31 | (0-65) | <0.0001 | 81% |
| IL-8 | 0 | (0-53) | 0 | (0-0) | 0.002 | 65% |
| IL-10 | 16 | (0-42) | 8 | (0-13) | 0.003 | 69% |
| sTNFR1 | 2708 | (1374-4175) | 820 | (510-1320) | <0.0001 | 88% |
| sTREM1 | 578 | (343-746) | 201 | (88-391) | <0.0001 | 80% |

TABLE 11
Breakdown of the ETC-19 triage decision
with and without adjudication

| | | Initial Triage Decision | | | |
|---|---|---|---|---|---|
| | | Discharge Home | | Admit to Hospital | |
| Adjudication | Avoidable | 98.7% | (152/154) | 8.4% | (14/166) |
| Results | Admission | | | | |
| | Required | 1.3% | (2/154) | 91.6% | (152/166) |
| | Hospitalization | | | | |

TABLE 12
Summary of SHAP values for respiratory
biomarkers in the ML model.

| | IL-6 | IL-8 | IL-10 | STNFR1 | sTREM |
|---|---|---|---|---|---|
| SHAP Values | 0.01099 | 0.015074 | 0.04633 | 0.298175 | 0.16291 |

1. A method for determining patient outcome risk in a patient with a respiratory illness, the method comprising:
a. obtaining a sample obtained from the patient;
b. quantitatively measuring in the sample a polypeptide level of one or more, preferably two or more, biomarkers selected from: sTNFR1, sTREM1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α or VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10; and
c. i) comparing the level of the one or more biomarkers in the sample with a control or cut-off level, wherein the differential level is indicative of patient outcome risk; or
ii) using the polypeptide level of several of the biomarkers in combination, as inputs for an algebraic calculation or machine learning model to determine patient outcome risk.

2. The method of claim 1 where respiratory illness is acute respiratory distress syndrome (ARDS) related to an infection.

3. The method of claim 2, wherein the infection is Influenza A, optionally influenza A is subtype H1N1.

4. The method of claim 2, wherein the infection is Influenza B.

5. The method of claim 2, wherein the infection is a coronavirus infection, optionally wherein the coronavirus is SARS-CoV, MERS-CoV or the coronavirus is SARS-nCoV-2019.

6. The method of claim 2, wherein the infection is a bacterial pneumonia.

7. The method of claim 1 wherein the respiratory illness is ARDS related to trauma.

8. The method of claim 2 where respiratory distress is ARDS related to exposure to an exogenous substance.

9. The method of any one of claims 1 to 8, wherein the sample is whole blood.

10. The method of any one of claims 1 to 8, wherein the sample is plasma.

11. The method of any one of claims 1 to 8, wherein the sample is serum.

12. The method of any one of claims 1 to 11, wherein the level of sTNFR1, sTREM1, and IL-6; sTNFR1, sTREM1, IL-6 and IL-8; or sTNFR1, sTREM1, IL-6, IL-8 and IL-10 is measured.

13. The method of any one of claims 1 to 11, wherein the level of at least 2 or 3 biomarkers is measured.

14. The method of any one of claims 1 to 11, wherein the level of at least 4 biomarkers is measured.

15. The method of any one of claims 1 to 11, wherein the level of at least 5 biomarkers is measured.

16. The method of any one of claims 1 to 15, wherein the method further comprises determining a CRB-65 score and using said score as a further input in the algebraic calculation or machine learning algorithm in determine the patient outcome risk.

17. The method of any one of claims 1 to 11, wherein the level of at least 2 biomarkers up to all of the biomarkers of the disclosure is measured.

18. The method of any one of claims 1 to 11, wherein the level of IL-6, IL-8, IL-10, sTREM1, sTNFR1 is measured.

19. The method of any one of claims 1 to 23, where the patient outcome risk is:
requirement of hospitalization or safe discharge,
requirement of mechanical ventilation,
requirement of treatment in an intensive care unit (ICU), and/or increased risk of death.

20. The method of claim 20, wherein the patient outcome risk is requirement for hospitalization or safe discharge and the method further comprises hospitalizing the patient or discharging the patient according to the patient outcome risk.

21. The method of claim 20, wherein the patient outcome risk is requirement for ventilation, and the method further comprises mechanically ventilating the patient.

22. The method of claim 20, wherein the patient outcome risk is requirement for treatment in the ICU and the method further comprises treating the patient in the ICU.

23. The method of any one of claims 1 to 22, wherein the sample is obtained from a patient that is hospitalized.

24. The method of claim 23, the sample obtained from the patient in hospital is obtained after the patient has a change in one or more symptoms of the respiratory illness.

25. The method of claim 24, wherein the change is amelioration of one or more symptoms of the respiratory illness and the patient is assessed for safe discharge.

26. The method of claim 24, wherein the change is worsening of one or more symptoms of the respiratory illness and the patient is assessed for requirement for mechanical ventilation or treatment in the ICU.

27. The method claim 25, wherein the method further comprises discharging the patient when the patient is determined to be safe to discharge or the method of claim 26 wherein the method further comprises mechanically ventilating the patient and/or treating the patient in the ICU when the patient is determined to require mechanical ventilation and/or ICU treatment.

28. A method for triaging a patient with a respiratory illness, the method comprising:
a. obtaining a sample obtained from the patient;
b. quantitatively measuring in the sample a polypeptide level of two or more biomarkers, the biomarkers comprising sTNFR1 and sTREM1, and optionally one or more of IL-6, IL-8 and IL-10, preferably wherein the two or more markers are sTNFR1, sTREM1, IL-6, IL-8 and IL-10; and
c. i) comparing the level of the two or more biomarkers in the sample with a control or cut-off level, wherein the differential level is indicative of patient outcome risk; or
ii) using the polypeptide level of several of the biomarkers in combination, as inputs for an algebraic calculation or machine learning model to determine whether the patient should be hospitalized or can be safely discharged.

29. The method of claim 28, where respiratory illness is acute respiratory distress syndrome (ARDS) related to an infection.

30. The method of claim 29, wherein the infection is Influenza A, optionally influenza A is subtype H1N1.

31. The method of claim 29, wherein the infection is Influenza B.

32. The method of claim 29, wherein the infection is a coronavirus infection, optionally wherein the coronavirus is SARS-CoV, MERS-CoV or the coronavirus is SARS-nCoV-2019.

33. The method of claim 29, wherein the infection is a bacterial pneumonia.

34. The method of claim 28 wherein the respiratory illness is ARDS related to trauma.

35. The method of claim 29, where respiratory distress is ARDS related to exposure to an exogenous substance.

36. The method of any one of claims 28 to 35, wherein the sample is whole blood.

37. The method of any one of claims 28 to 35, wherein the sample is plasma.

38. The method of any one of claims 28 to 35, wherein the sample is serum.

39. The method of any one of claims 28 to 38, wherein the level of sTNFR1, sTREM1, and IL-6; sTNFR1, sTREM1, IL-6 and IL-8; or sTNFR1, sTREM1, IL-6, IL-8 and IL-10 is measured.

40. The method of any one of claims 28 to 38, wherein the level of at least 3 biomarkers is measured.

41. The method of any one of claims 28 to 38, wherein the level of at least 4 biomarkers is measured.

42. The method of any one of claims 28 to 38, wherein the level of at least 5 biomarkers is measured.

43. The method of any one of claims 28 to 38, wherein the method further comprises determining a CRB-65 score and using said score as a further input in the algebraic calculation or machine learning algorithm in determining whether the patient should be hospitalized or can be safely discharged.

44. The method of any one of claim 28 to 38 or 43, wherein the level of at least 2 biomarkers up to all of the biomarkers of the disclosure is measured.

45. The method of any one of claim 28 to 38 or 43, wherein the level of IL-6, IL-8, IL-10, sTREM1, sTNFR1 is measured.

46. The method of any one of claims 28 to 45, wherein the method further comprises hospitalizing the patient or discharging the patient.

47. The method of any one of claims 1 to 46, wherein the sample is obtained upon clinical presentation, optionally at an emergency room or urgent care centre.

48. The method of any one of claims 1 to 47, wherein the sample is obtained from a patient in hospital.

49. The method of any one of claims 1 to 48, wherein the polypeptide level of one or more, preferably two or more, biomarkers is measured using a multiplex assay, optionally a 5-plex assay.

50. The method of any one of claims 1 to 49, wherein the quantitatively measuring comprises the steps of incubating the sample with a detection agent for each of the one or more, preferably two or more, biomarkers; obtaining signal intensities for each of the one or more, preferably two or more, biomarkers, processing the signal intensities to calculate concentrations of the one or more, preferably two or more, biomarkers in the sample, wherein the concentrations are compared or used as inputs in step c).

51. The method of any one of claims 1 to 50, wherein the machine learning model comprises a decision tree.

52. The method of any one of claims 1 to 51, wherein the polypeptide level is measured using an assay with a limit of detection for each of one or more, preferably two or more, biomarkers, wherein the lower limit of detection (LLOD) at least 1 pg/mL for IL-6, IL-8, and/or IL-10 and at least 15 pg/mL for sTNFR1 and/or sTREM1, optionally wherein the LLOD for IL-6 is at least 21 pg/mL, for IL-8 is at least 27 pg/mL, for IL-10 is at least 7 pg/mL, for sTNFR1 is at least 17 pg/mL, and for sTREM1 is at least 44 pg/mL.

53. The method of any one of claims 1 to 52, wherein the relative feature importance of the biomarkers of the machine learning model can be given by their Shapley Additive Explanations (SHAP) values.

54. Use of the method of any one of claims 1 to 27 and 47 to 53 for screening or stratifying patients as less or more likely to require hospitalization, mechanical ventilation and/or ICU treatment or the method of any one of claims 28 to 53 for screening patients as less or more likely to require hospitalization or to be less or more likely to be safely discharged.

55. A kit or immunoassay comprising at least a detection antibody specific for sTNFR1 and a detection antibody specific for sTREM1 and optionally one or more other detection antibodies each specific for a biomarker selected from IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α and VEGF,

56. The kit or immunoassay of claim 55, wherein the one or more detection antibodies comprise antibodies specific for IL-6, IL-8 and IL-10.

57. The kit or immunoassay of claim 55 or 56 wherein the detection antibodies are coupled to beads and/or labelled.

58. The kit or immunoassay of any one of claims 55 to 57 further comprising one or more of a 96-well plate, optionally wherein the detection antibodies are fixed, standards, assay buffer, wash buffer, sample diluent, standard diluent, detection antibody diluent, streptavidin-PE, a filter plate or sealing tape.

59. The kit or immunoassay of any one of claims 55 to 58, for performing the method of any one of claims 1 to 53.

60. A computer-implemented method for determining patient outcome risk in a patient with a respiratory illness, the method comprising:
a. obtaining a polypeptide level of one or more, preferably two or more, biomarkers selected from: sTNFR1 and sTREM-1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α and VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10;
b. calculating patient outcome risk with a machine learning model using the received polypeptide levels as inputs.

61. The computer implemented method of claim 60 where respiratory illness is acute respiratory distress syndrome (ARDS) related to an infection.

62. The computer implemented method of claim 61, wherein the infection is Influenza A, optionally influenza A is subtype H1N1.

63. The computer implemented method of claim 61, wherein the infection is Influenza B.

64. The computer implemented method of claim 61, wherein the infection is a coronavirus infection, optionally wherein the coronavirus is SARS-CoV, MERS-CoV or the coronavirus is SARS-nCoV-2019.

65. The computer implemented method of claim 61, wherein the infection is a bacterial pneumonia.

66. The computer implemented method of claim 60 wherein the respiratory illness is ARDS related to trauma.

67. The computer implemented method of claim 60 where respiratory distress is ARDS related to exposure to an exogenous substance.

68. The computer implemented method of any one of claims 60 to 67 where the patient outcome risk is:
requirement of hospitalization,
requirement of mechanical ventilation,
requirement of treatment in the intensive care unit (ICU), and/or increased risk of death.

69. The computer implemented method of any one of claims 60 to 68, wherein the step of obtaining a polypeptide level method further includes the step of:
quantitatively measuring a polypeptide level of one or more, preferably two or more, biomarkers of a sample obtained from a patient.

70. The computer implemented method of any one of claims 60 to 69, wherein the biomarkers selected include IL-6, IL-8, IL-10, sTNFR1 and sTREM1, and optionally comprises using a CRB-65 score as an input.

71. The method of any one of claims 1 to 53 or the computer implemented method of claim 70, wherein the machine learning model comprises a decision tree.

72. The method of any one of claims 1 to 53 or the computer implemented method of claim 71, wherein the relative feature importance of the biomarkers of the machine learning model can be given by their Shapley Additive Explanations (SHAP) values.

73. A system for determining patient outcome risk in a patient with a respiratory illness, the system comprising:
a processor; and
at least one non-transitory memory containing instructions which when executed by the processor cause the system to:
obtain a polypeptide level of one or more, preferably two or more, biomarkers selected from: sTNFR1 and sTREM-1, IL-6, IL-8, IL-10, IL-1RA, IL-2, IL-4, IL-7, IL-9, IL-13, IL-17, IFN-g, IP-10, MCP-1, G-CSF, GM-CSF, FGF-basic, SCGF-β, GRO-α, MIP1-α, MIP1-β, CK-18, PDGF-bb, caspase 3, HMGB-1, TNF α, and VEGF, preferably wherein the two or more biomarkers are sTNFR1, sTREM1, and optionally one or more of IL-6, IL-8 and IL-10, more preferably wherein the two or more markers are sTNFR1, sTREM1, IL-6, IL-8 and IL-10, and optionally a CRB-65 score; and
calculate patient outcome risk with a machine learning model using the received polypeptide levels and optionally CRB-65 score as inputs.
